(19) United States
(12) Patent Application Publication
(10) Pub. No.: US 2009/0093749 A1
(43) Pub. Date: Apr. 9, 2009

(54) APPARATUS AND METHOD FOR SKIN TREATMENT

(76) Inventors: Pinchas SHALEV, Herzliya (IL); Zion AZAR, Shoham (IL)

Correspondence Address: DANIEL J SWIRSKY, 55 REUVEN ST., BEIT SHEMESH, 99544, IL

(21) Appl. No.: 12/324,877

(22) Filed: Nov. 27, 2008

Related U.S. Application Data
(62) Division of application No. 11/955,416, filed on Dec. 13, 2007.
(60) Provisional application No. 60/869,767, filed on Dec. 13, 2006.

(51) Int. Cl. A61N 1/18; A61N 1/00

(52) U.S. Cl. 604/20; 607/115

(57) ABSTRACT

A device for treatment of skin tissue includes RF electrodes for applying RF energy to the skin and a dispensing unit for dispensing a depilatory substance onto the skin. The device may include a housing, an RF electrode assembly including RF electrodes for applying RF energy to the skin, one or more of the RF electrodes is movable relative to the housing and to the surface of the skin for distributing the RF energy to various parts of the skin and for spreading the depilatory substance along the surface of the skin. The RF electrodes are energized by an internal or external RF energy source. A method for treatment of skin tissue includes applying a depilatory substance to the skin and applying RF energy to the depilatory substance treated skin.

### CROSS-REFERENCE TO RELATED US APPLICATIONS

This application is a divisional application of U.S. patent application Ser. No. 11/955,416, filed Dec. 13, 2007, which claims priority from and the benefit of U.S. Provisional Patent Application Ser. No. 60/869,767, filed on Dec. 13, 2006, entitled “Apparatus and Method for Skin Treatment,” all of which applications are incorporated herein by reference in their entirety.

### FIELD OF THE INVENTION

This invention relates in general to the fields of hair removal and skin treatment and more particularly to devices systems and methods combining the application of a depilatory agent to the skin with RF current induced heat treatment of the skin.

### BACKGROUND OF THE INVENTION

The removal of unwanted facial and body hair can be accomplished with non-mechanized means, for example razors, tweezers or wax, all of which are uncomfortable to use, irritate the skin and/or cause damage to the skin.

Mechanized cutting means for cutting hair, for example dry shavers, in addition to being uncomfortable to use, are limited to cutting hair of a specific length. Beard trimmers, for example, cut facial hair stubble, but cannot cut longer hairs on the scalp.

The use of heated wires or other structures to cut hair from a skin surface has been proposed. However, a heat generator that generates heat of a sufficient magnitude to cut hair and that cuts the hair close to the skin, often damages the skin. Alternatively, since the heat generator is offset from the skin to prevent skin damage, unwanted stubble is left behind.

U.S. Pat. No. 3,934,115 to Peterson, discloses the use of parallel metal strips on the upper side of a ceramic facing that contacts the skin, are used to cut hair. U.S. Pat. No. 2,727,132 to Hills and Italian Patent IT 1201364P to Massimo disclose a continuously heated element for burning hair. U.S. Pat. No. 558,465, to Bell and U.S. Pat. No. 589,445, to D. Seide, U.S. Pat. No. 2,727,132 to G. S. Hills, U.S. Pat. No. 3,093,724 to G. L. Johnson, U.S. Pat. No. 5,064,993 to Hashimoto and U.S. Pat. No. 6,307,181 to Hashimoto, French Patent FR 2531655 to F. Solvinto, European Patent EP 0201189 to F. Solvinto, and French Patent No. FR 2612381 to E. Michit, all disclose a continuously heated wire for burning hair. U.S. Pat. No. 3,474,224 to J. F. Carter, discloses a circular comb device for burning nose hairs. Aside from physically separating the skin from the heated element, the above cited references do not appear to provide other protection against burning of the skin.

All the above devices and methods are in use for short term hair removal and do not provide permanent hair reduction.

Other types of devices are directed to long term hair removal. Electrolysis devices are based on the use of “electric needles”. Such fine needles are inserted into the hair follicle and apply an electric current to each hair. The current heats the hair and causes its carbonization and also heats the tissue near the hair causing its coagulation and partial or full coagulation of the blood capillaries which supply blood to the hair follicle. While such devices can result in permanent hair removal, each hair must be treated individually, making hair removal by this method a tedious often painful, time consuming, and expensive.

Another class of known devices includes photothermolysis devices which are usually operated by physicians in clinics. These devices are based either on lasers (e.g. Ruby lasers) such as the laser device disclosed in U.S. Pat. No. 5,059,192 to Zaias or on an incoherent light source coupled with filters and elaborate electronics to provide pulses of various durations and wave lengths as described in U.S. Pat. No. 5,405,368 to Eckhouse and in European Patent publications EP 0 788 814 and EP 0736308. The above referenced Eckhouse patents and European Publications disclose heating the hair directly by a high flux of visible radiation that is absorbed by the hair follicles. Various filters and/or pulse lengths are used depending on the depth of penetration desired and the color of the hair being removed.

Another type of long term hair removal method, disclosed in U.S. Pat. No. 6,702,808 is based on using RF energy coupled with a light energy that provides selective pre-heating of the hair to be destroyed. Light energy is applied to the skin and selectively pre-heats the hair follicle above the temperature of the surrounding skin but below the coagulation temperature, RF energy is then applied to the skin to cause coagulation of the hair follicle since the applied RF energy causes more heating of the heated hair follicle than the surrounding skin. In order to achieve selective pre-heating of the hair follicle, the color of the hair should be darker than the surrounding skin. Thus this method suffers from the same deficiencies as Photothermolysis devices.

Other devices and methods for applying RF energy to the skin include, inter alia, Published International Patent Application, International Publication Number WO 2007/088541 to Azar et al., incorporated herein by reference in its entirety, which discloses apparatus for hair removal using a combination of a hot cutting element and an RF energy source for removing hair, and co-pending U.S. patent application Ser. No. 11/828,371 to Azar et al., filed Jul. 26, 2007 and entitled “APPARATUS AND METHOD FOR NON-INVASIVE TREATMENT OF SKIN TISSUE” which discloses a device for skin treatment having multiple RF electrodes.

Methods for hair removal using application of various different depilatory compositions and substances are well known in the art. Such methods of chemical depilation involve application of depilatory creams, liquids, foams or ointments to the surface of the skin and hairs, waiting a certain period of time for the depilatory composition to affect the hairs and wiping or removing or washing away the depilatory composition. Such depilatory compositions and substances may include, inter alia, active ingredients such as but not limited to potassium thioglycolate, other thioglycolates, sulphhydryl group (SH group) containing compounds, calcium hydroxide and other different active ingredients. Such depilatory compositions may also include different various vehicles, cream bases, solvents, emulsifiers, skin emollients, pH adjusting substances, perfume and the like.

The chemical composition, formulation and preparation of such depilatory substances and/or depilatory compositions and formulation is well known in the art, is not the subject matter of the present invention and is therefore not described in detail hereinafter.

Typically, when depilation is desired, the depilatory composition is applied to a region of the skin in the form of a liquid, spray, ointment, cream, foam, or the like. After a certain period of time, the depilatory substance is removed from the skin by washing or by other methods, to avoid undesirable effects on the skin such as, rash, excessive keratinolytic effects, and other side effects of hair removal.

U.S. Pat. No. 6,533,775 to Rizoiu incorporated herein by reference in its entirety, discloses a shaving device including a depilatory cream reservoir, a light source for applying a beam of diffused light to the skin, and a blade for removing the hairs. The Rizoiu device has certain disadvantages which prevent efficient use thereof for shaving and hair removal. One problem is that most commercially available efficient depilatory compositions are opaque to visible light. Another disadvantage is that the light energy emission assembly used for diffusing the light onto the skin is cumbersome and may require the use of mirrors, beams splitters, light guides, light diffusers and/or other optical assemblies that may be required for delivering the light to the surface of the skin. Such optical devices may be expensive and cumbersome. Yet another problem is that in order to absorb the light certain photosensitive components may have to be added to the depilatory composition or the wavelength of the light may have to be matched to the wavelength to which the cream is sensitive. In other words, the efficiency of light absorption may depend on the particular wavelength of light used and on the absorbance of the light by the cream or by the cream components being used. An additional problem with the Rizoiu device is that the blade included in the Rizoiu device may mechanically disrupt, nick or tear the skin tissues that were softened by the chemical and keratinolytic action of the depilatory cream, causing pain and/or discomfort to the user and may result in infection of cut skin regions.

### SUMMARY OF THE INVENTION

There is therefore provided a device for treatment of skin tissue. The device includes RF electrodes for applying RF energy to the skin and a dispensing unit for dispensing a depilatory substance onto the skin.

Furthermore, in accordance with an embodiment of the device, the device includes an RF energy source for energizing said RF electrodes.

Furthermore, in accordance with an embodiment of the device, the RF energy source may be an RF energy source included in the device or an RF energy source disposed outside the device and electrically coupled to the R-F electrodes.

Furthermore, in accordance with an embodiment of the device the device further including a hand held housing, wherein the RF energy source, the RF electrodes and the dispensing unit are attached to the housing.

Furthermore, in accordance with an embodiment of the device, the device further includes a hand held housing which is a part of a system including an RF current generating unit external to said the and electrically couplable to the RF electrodes for providing RF energy thereto. The RF electrodes and the dispensing unit are attached to the hand held housing.

Furthermore, in accordance with an embodiment of the device, the RF electrodes are selected from fixed (stationary) RF electrodes and movable RF electrodes.

Furthermore, in accordance with an embodiment of the device, the RF electrodes are RF electrodes movable relative to the device and to the surface of the skin for distributing the RF energy to various parts of the skin and for spreading the depilatory substance on the surface of the skin.

Furthermore, in accordance with an embodiment of the device the RF electrodes are attached to an RF electrode assembly.

Furthermore, in accordance with an embodiment of the device, the RF electrode assembly includes a motor for moving at least one RF electrode of the RF electrodes.

Furthermore, in accordance with an embodiment of the device, the motor is an electrical motor.

Furthermore, in accordance with an embodiment of the device the RF electrodes are attached to a movable RF electrode carrying member coupled to the motor.

Furthermore, in accordance with an embodiment of the device, the device also includes a stabilizing member attached to the housing for stabilizing the position of the device on the skin while the RF electrode carrying member is moving along the surface of the skin.

Furthermore, in accordance with an embodiment of the device, the stabilizing member has a first portion attached to the housing and a second portion configured for contacting the skin.

Furthermore, in accordance with an embodiment of the device, the second portion of the stabilizing member has multiple spaced apart skin contacting portions for contacting the skin.

Furthermore, in accordance with an embodiment of the device, the stabilizing member is detachably attached to the housing.

Furthermore, in accordance with an embodiment of the device, the motor has a rotatable shaft and the RF electrode carrying member is eccentrically attached to the shaft.

Furthermore, in accordance with an embodiment of the device, the RF electrode carrying member is eccentrically attached to a coupling member attached to the rotatable shaft.

Furthermore, in accordance with an embodiment of the device, the motor has a rotatable shaft having a first shaft portion proximal to the motor and a second shaft portion bent at an angle relative to the first shaft portion of the shaft and the RF electrode carrying member is movably attached to the second shaft portion.

Furthermore, in accordance with an embodiment of the device, the second shaft portion has a spherically shaped member formed at its end distal from said motor. The RF electrode carrying member has a spherically shaped socket formed therein. The spherically shaped member is disposed within the spherically shaped socket to enable the RF electrode carrying member to move at an angle to said second shaft portion.

Furthermore, in accordance with an embodiment of the device, the second shaft portion is movably disposed within a recess formed within the RF electrode carrying member.

Furthermore, in accordance with an embodiment of the device, the RF electrode carrying member is movably attached to the RF electrode assembly or to the housing by one or more spring-like elements.

Furthermore, in accordance with an embodiment of the device, the spring-like members are selected from a plurality of springs, a plurality of elastic members, and a flexible sleeve-like member.

Furthermore, in accordance with an embodiment of the device, the flexible sleeve-like member is a concertina-like member.

Furthermore, in accordance with an embodiment of the device, the spring-like member(s) is a flexible sleeve-like member having a first end attached to the housing or to the RF electrode assembly and a second end attached to the RF electrode carrying member.

Furthermore, in accordance with an embodiment of the device, the spring-like member(s) are electrically conducting elements electrically connected to the RF electrodes for providing RF energy to said RF electrodes.

Furthermore, in accordance with an embodiment of the device, the dispensing unit is selected from a hand operated dispensing unit, a pump operated dispensing unit, a valve operated dispensing unit, a pressurized dispensing unit, a propellant operated dispensing unit, a disposable dispensing unit, a replaceable dispensing unit, a refillable dispensing unit, a canister based dispensing unit, a dispensing unit including a fixed reservoir, a dispensing unit including a replaceable reservoir, and a dispensing unit including a disposable reservoir.

Furthermore, in accordance with an embodiment of the device, the dispensing unit is fluidically coupled to one or more hollow conduits passing through one or more of the RF electrodes for applying the depilatory substance to the skin through the one or more conduits.

Furthermore, in accordance with an embodiment of the device, the dispensing unit is fluidically coupled to one or more hollow conduits passing through an RF electrode carrying member to which said RF electrodes are attached, for applying the depilatory substance to the skin through the conduit(s).

Furthermore, in accordance with an embodiment of the device, the dispensing unit includes a replaceable squeezable reservoir containing the depilatory substance and a pressure exerting mechanism configured for applying pressure on the squeezable reservoir.

Furthermore, in accordance with an embodiment of the device, the pressure exerting mechanism includes a compartment for accommodating the reservoir and a spring loaded pressure plate configured for applying pressure on the squeezable reservoir.

Furthermore, in accordance with an embodiment of the device, the squeezable reservoir is a squeezable bag made from a pliable material.

Furthermore, in accordance with an embodiment of the device, the pliable material is a polymer based pliable material.

Furthermore, in accordance with an embodiment of the device, the squeezable reservoir also includes a dispensing tube configured to be sealingly attached to a depilatory substance inlet port included in the dispensing unit.

Furthermore, in accordance with an embodiment of the device, the device includes an RF electrode carrying member to which said RF electrodes are attached, and the squeezable reservoir also includes an elongated flexible hollow dispensing tube configured to be inserted through a hollow guiding member attached to the RF electrode carrying member. The dispensing tube is capable of being inserted into said guiding member and reaching an opening on the surface of said RF electrode carrying member facing said skin wherein said depilatory substance may be applied to the skin through an opening at the end of said dispensing tube.

Furthermore, in accordance with an embodiment of the device, the dispensing tube further includes a controllably openable valve for controlling the dispensing of said depilatory substance through said dispensing tube.

Furthermore, in accordance with an embodiment of the device, the dispensing tube is an elastic member having a lumen in fluidic communication with the squeezable reservoir and the device also includes a mechanism for constricting or closing the lumen of the dispensing tube by applying pressure on the dispensing tube to regulate or terminate the passage of depilatory substance member through said lumen.

Furthermore, in accordance with an embodiment of the device, the device also includes at least one temperature sensor for determining the temperature of the skin.

Furthermore, in accordance with an embodiment of the device, the temperature sensor is selected from a remote sensing sensor which does not contact the skin, a contact type sensor operable in contact with the skin and any combinations thereof.

Furthermore, in accordance with an embodiment of the device, the temperature sensor is selected from an infra-red detecting temperature sensor, a thermistor based temperature sensor and any combinations thereof.

Furthermore, in accordance with an embodiment of the device, the device also includes at least one temperature sensor for determining the temperature of one or more of the RF electrodes.

Furthermore, in accordance with an embodiment of the device, the temperature sensor(s) is a thermistor.

There is also provided, in accordance with an embodiment of the device, a device for treatment of skin tissue. The device includes means for applying RF energy to the skin and means for applying a depilatory substance onto the skin.

There is also provided, in accordance with an embodiment of the device, a device for applying RF energy to skin tissue treated with a depilatory substance, the device includes a housing, an RF electrode assembly attached to the housing and including RF electrodes for applying RF energy to the skin, at least one of the RF electrodes is movable relative to the housing and to the surface of the skin for distributing the RF energy to various parts of the skin and for spreading the depilatory substance along the surface of the skin. The device also includes an RF energy source for energizing the RF electrodes.

Furthermore, in accordance with an embodiment of the device, the RF energy source is selected from an RF energy source disposed within the housing and electrically connectable to the RF electrodes, and an RF energy source disposed outside of the housing and electrically connectable to the RF electrodes.

Furthermore, in accordance with an embodiment of the device, the RF electrode assembly includes a motor for moving the at least one RF electrode.

Furthermore, in accordance with an embodiment of the device, the motor is an electrical motor.

Furthermore, in accordance with an embodiment of the device, the RF electrodes are attached to a movable RF electrode carrying member coupled to the motor.

Furthermore, in accordance with an embodiment of the device, the motor has a rotatable shaft and the RF electrode carrying member is eccentrically attached to the shaft.

Furthermore, in accordance with an embodiment of the device, the motor has a rotatable shaft having a first shaft portion proximal to the motor and a second shaft portion bent at an angle relative to the first shaft portion and wherein the RF electrode carrying member is attached to the second shaft portion.

Furthermore, in accordance with an embodiment of the device, the second shaft portion has a spherically shaped member formed at its end distal from the motor. The RF electrode carrying member has a spherically shaped socket formed therein. The spherically shaped member is disposed within the spherically shaped socket to enable the RF electrode assembly to move at an angle to the second shaft portion.

Furthermore, in accordance with an embodiment of the device, the electrode carrying member is movably attached to the RF electrode assembly or to the housing by one or more spring-like members.

Furthermore, in accordance with an embodiment of the device, the one or more spring-like members are selected from a plurality of springs, a plurality of elastic members, and a flexible sleeve-like member.

Furthermore, in accordance with an embodiment of the device, the flexible sleeve-like member is a concertina-like member.

Furthermore, in accordance with an embodiment of the device, the one or more spring-like members is a flexible sleeve like member. The flexible sleeve like member has a first end sealingly attached to the housing or to the RF electrode assembly. The flexible sleeve-like member has a second end sealingly attached to the RF electrode carrying member.

Furthermore, in accordance with an embodiment of the device, the one or more spring-like members is a flexible sleeve-like member. The flexible sleeve like member has a first end attached to the housing or to the RF electrode assembly and a second end attached to the RF electrode carrying member.

Furthermore, in accordance with an embodiment of the device, the one or more spring-like members are electrically conducting elements electrically connected to the RF electrodes for providing RF energy to the RF electrodes.

Furthermore, in accordance with an embodiment of the device, the device also includes a stabilizing member attached to the housing for stabilizing the position of the device on the skin while the RF electrode carrying member is moving along the surface of the skin.

Furthermore, in accordance with an embodiment of the device, the stabilizing member has a first portion attached to the housing and a second portion configured for contacting the skin.

Furthermore, in accordance with an embodiment of the device, the second portion of the stabilizing member has a plurality of spaced apart skin contacting portions for contacting the skin.

Furthermore, in accordance with an embodiment of the device, the stabilizing member is detachably attached to the housing.

Furthermore, in accordance with an embodiment of the device, the device also includes at least one dispensing unit for dispensing a depilatory substance onto the skin.

Furthermore, in accordance with an embodiment of the device, the at least one dispensing unit is selected from a hand operated dispensing unit, a pump operated dispensing unit, a valve operated dispensing unit, a pressurized dispensing unit, a propellant operated dispensing unit, a disposable dispensing unit, a replaceable dispensing unit, a refillable dispensing unit, a canister based dispensing unit, a dispensing unit including a fixed reservoir, a dispensing unit including a replaceable reservoir, and a dispensing unit including a disposable reservoir and any combinations thereof.

Furthermore, in accordance with an embodiment of the device, the at least one dispensing unit includes a replaceable squeezable reservoir including the depilatory substance and a pressure exerting mechanism configured for applying pressure on the squeezable reservoir.

Furthermore, in accordance with an embodiment of the device, the pressure exerting mechanism includes a compartment for accommodating the squeezable reservoir and a spring loaded pressure plate configured for applying pressure on the squeezable reservoir.

Furthermore, in accordance with an embodiment of the device, the squeezable reservoir is a squeezable bag made from a pliable material.

Furthermore, in accordance with an embodiment of the device, the pliable material is a polymer based pliable material.

Furthermore, in accordance with an embodiment of the device, the pliable material is polyethylene.

Furthermore, in accordance with an embodiment of the device, the squeezable reservoir also includes a dispensing tube configured to be sealingly attached to a depilatory substance inlet port included in the dispensing unit.

Furthermore, in accordance with an embodiment of the device, the device includes an RF electrode carrying member to which the RF electrodes are attached, and wherein the squeezable reservoir also includes an elongated flexible hollow dispensing tube configured to be inserted through a hollow guiding member attached to the RF electrode carrying member. The dispensing tube is capable of being inserted into the guiding member and reaching an opening on the surface of the RF electrode carrying member facing the skin. The depilatory substance may be applied to the skin through an opening at the end of said dispensing tube.

Furthermore, in accordance with an embodiment of the device, the dispensing tube further includes a controllably openable valve for controlling the dispensing of the depilatory substance through the dispensing tube.

Furthermore, in accordance with an embodiment of the device, the dispensing tube is an elastic member having a lumen in fluidic communication with the squeezable reservoir. The device also includes a mechanism for constricting or closing the lumen of the dispensing tube by applying pressure on the dispensing tube to regulate or terminate the passage of the dispensing member through the lumen.

Furthermore, in accordance with an embodiment of the device, the dispensing unit is fluidically coupled to one or more hollow conduits passing through one or more of the RF electrodes for applying the depilatory substance to the skin through the one or more conduits.

Furthermore, in accordance with an embodiment of the device, the dispensing unit is fluidically coupled to one or more hollow conduits passing through an RF electrode carrying member to which the RF electrodes are attached, for applying the depilatory substance to the skin through the one or more conduits.

Furthermore, in accordance with an embodiment of the device, the device also includes at least one temperature sensor for determining the temperature of the skin.

Furthermore, in accordance with an embodiment of the device, the temperature sensor(s) are selected from a remote sensing sensor which does not contact the skin, a contact type sensor operable in contact with the skin and any combinations thereof.

Furthermore, in accordance with an embodiment of the device, the temperature sensor(s) are selected from an infra-red detecting temperature sensor, a thermistor based temperature sensor and any combinations thereof.

Furthermore, in accordance with an embodiment of the device, the device also includes at least one temperature sensor for determining the temperature of one or more of the RF electrodes.

Furthermore, in accordance with an embodiment of the device, the at least one temperature sensor is a thermistor.

There is also provided a method for treatment of skin tissue, the method comprising the steps of, applying a depilatory substance to the skin, and applying RF energy to the skin through RF electrodes for heating the skin and the depilatory substance.

Furthermore, in accordance with an embodiment of the method, the method also includes the step of sensing the temperature of at least one of the RF electrodes and terminating the step of applying RF energy to the skin if the temperature of at least one of the RF electrodes exceeds a temperature threshold value.

Furthermore, in accordance with an embodiment of the method, the method further includes the step of terminating the applying to the skin of the depilatory substance if the temperature of at least one of the RF electrodes exceeds a temperature threshold value.

Furthermore, in accordance with an embodiment of the method, the method also includes the step of sensing the temperature of the skin and terminating the step of applying RF energy to the skin if the temperature of the skin exceeds a temperature threshold value.

Furthermore, in accordance with an embodiment of the method, the method also includes the step of terminating the applying to the skin of the depilatory substance if the temperature of the skin exceeds a temperature threshold value.

Furthermore, in accordance with an embodiment of the method, the method also includes the step of sensing the temperature of at least one of the RF electrodes and providing an indication if the temperature of the RF electrode(s) exceeds a temperature threshold value.

Furthermore, in accordance with an embodiment of the method, the indication is selected from a visual signal, an auditory signal and combinations thereof.

Furthermore, in accordance with an embodiment of the method, the method also includes the step of sensing the temperature of the skin and providing an indication if the temperature of the skin exceeds a temperature threshold value.

Furthermore, in accordance with an embodiment of the method, the indication is selected from a visual signal, an auditory signal and combinations thereof.

Furthermore, in accordance with an embodiment of the method, the method also includes the step of moving the RF electrodes along said skin.

Furthermore, in accordance with an embodiment of the method, the RF electrodes are attached to a motorized movable RF electrode carrying member, wherein the step of moving the RF electrodes comprises automatically moving the movable RF electrode carrying member.

Furthermore, in accordance with an embodiment of the method, the movable RF electrode carrying member is a rotatable member, wherein the step of automatically moving comprises automatically rotatingly moving the movable RF electrode carrying member.

Furthermore, in accordance with an embodiment of the method, the method also includes the step of sensing the temperature of the skin and terminating the moving of the RF electrodes along the skin if the temperature of the skin exceeds a temperature threshold value.

Furthermore, in accordance with an embodiment of the method, the step of moving comprises automatically moving the RF electrodes along the skin, wherein the method also includes the step of automatically terminating the applying of RF energy to the skin and the moving of the RF electrodes along the skin if the temperature of the skin exceeds a temperature threshold value.

Furthermore, in accordance with an embodiment of the method, the method also includes the step of sensing the temperature of at least one of the RF electrodes and automatically terminating the applying of RF energy to the skin and the moving of the RF electrodes along the skin if the temperature of at least one of the RF electrodes exceeds a temperature threshold value.

Furthermore, in accordance with an embodiment of the method, the step of moving comprises automatically moving said RF electrodes along the skin, wherein the method also includes the step of automatically terminating the applying of RF energy to the skin and the moving of the RF electrodes along the skin if the temperature of at least one of the RF electrodes exceeds a temperature threshold value.

Furthermore, in accordance with an embodiment of the method, 95. the step of applying a depilatory substance to the skin is selected from,

manually dispensing the depilatory substance onto the skin,

controllably dispensing the depilatory substance onto the skin, and

automatically dispensing the depilatory substance onto the skin.

There is also provided a method for treatment of skin tissue. The method includes the steps of applying to the skin a depilatory substance, and applying RF energy to the skin for selectively heating skin tissues in the vicinity of hair shafts of the skin to selectively increase the effect of the depilatory substance on the skin tissues.

There is finally provided a method for treatment of skin tissue. The method includes the steps of applying to the skin a depilatory substance, and applying RF energy to the skin for preferentially heating at least the skin tissues in the vicinity of hair shafts of the skin and the hair shafts, to selectively increase the effect of the depilatory substance on at least one of the skin tissues and the hair shafts.

### BRIEF DESCRIPTION OF THE DRAWINGS

The invention is herein described, by way of example only, with reference to the accompanying drawings, in which like components are designated by like reference numerals, wherein:

FIGS. 1-2 are photographs illustrating the results of experiments for removing hair by using a chemical depilatory composition alone as compared to using the combination of a chemical depilatory composition and heating the skin by direct application of RF currents, in accordance with an embodiment of a skin treating method of the present application;

FIGS. 3 and 4 are photomicrographs illustrating the skin region surrounding different human hairs after application of a chemical depilatory composition without heating of the skin (FIG. 4) and after combined application of a chemical depilatory composition with RF heating of the skin (FIG. 5), in accordance with an embodiment of a skin treating method of the present application;

FIG. 5 is a photomicrograph illustrating selective damage of the skin regions surrounding human hairs two days after application of a depilatory composition combined with RF heating of the skin, in accordance with an embodiment of the a skin treating method of the present application;

FIG. 6 is a photomicrograph illustrating a region of skin demonstrating damage caused to the skin regions surrounding the hairs after treatment of the skin with a chemical depilatory composition combined with skin heating using the application of RF currents to the skin, in accordance with an embodiment of a skin treating method of the present application;

FIG. 7 is an isometric view schematically illustrating an exemplary RF current applicator system useful for applying RF currents to the skin in the presence of depilatory substances or compositions in accordance with an embodiment of the skin treating systems of the present application;

FIG. 8 is a schematic part cross-sectional diagram illustrating a device having a replaceable RF electrode assembly and a reservoir for applying a depilatory substance or composition to the skin and for applying RF energy to the depilatory substance treated skin, in accordance with an embodiment of the skin treating devices of the present application;

FIG. 9 is a schematic front view of the device illustrated in FIG. 8;

FIG. 10 is a schematic part cross-sectional diagram illustrating a device having a refillable reservoir for applying a depilatory substance or composition to the skin and for applying RF energy to the depilatory substance treated skin, in accordance with an embodiment of the skin treating devices of the present application;

FIG. 11 is a schematic front view of the device illustrated in FIG. 10;

FIG. 12 is a schematic part cross-sectional diagram illustrating a device for applying RF energy to skin treated with a depilatory substance. The device has an infrared thermometer for monitoring skin temperature, in accordance with an embodiment of the skin treating devices of the present application;

FIG. 13 is a schematic front view of the device illustrated in FIG. 12;

FIG. 14 is a schematic part cross-sectional diagram illustrating a device having a replaceable RF electrode assembly having a passage passing therethrough and a reservoir for applying a depilatory substance or composition to the skin and for applying RF energy to the depilatory substance treated skin, in accordance with an embodiment of the skin treating devices of the present application;

FIG. 15 is a schematic part cross-sectional part isometric side view illustrating a device having a hand operated pumping dispenser for dispensing depilatory substance and a motorized movable RF electrode assembly, in accordance with an embodiment of the skin treating devices of the present application;

FIG. 16 is a schematic side view illustrating the motorized RF electrode assembly of the device of FIG. 15.

FIG. 17 is a schematic exploded isometric view of a skin treatment device having a stabilizing member and a flexible RF electrode assembly protecting sleeve member, in accordance with an embodiment of the skin treating devices of the present application;

FIG. 18 is a schematic isometric view illustrating the skin treatment device of FIG. 18 without the stabilizing member and the flexible electrode assembly protecting sleeve;

FIG. 19 is a schematic cross-sectional diagram illustrating in detail the motorized movable RF electrode assembly of the skin treatment device of FIG. 18;

FIG. 20 is a schematic cross-sectional diagram illustrating part of a skin treating device having a disposable depilatory substance reservoir, in accordance with an embodiment of the skin treating devices of the present application;

FIGS. 21 and 22 are photographs illustrating differences in appearance of human skin after two different skin treatment methods were applied to the skin; and

FIG. 23 is a schematic flow diagram illustrating the steps of a method of hair removal and/or skin treatment combining the application of a depilatory composition to the skin with the application of RF currents to the treated skin region;

FIG. 24 is a schematic graph illustrating a curve representing the skin temperature as a function of time during various stages of operation of skin treating devices and systems of the present application; and

FIG. 25 which is a schematic part cross-sectional diagram illustrating in detail another motorized movable RF electrode assembly having movable and stationary RF electrodes, suitable for use with the skin treatment device of FIG. 18.

### DETAILED DESCRIPTION OF THE INVENTION

### Notation Used Throughout

The following notation is used throughout this document.

| Term | Definition |
|---|---|
| AC | Alternating current |
| DC | Direct current |
| dpi | Dots per inch |
| EM | Electromagnetic |
| GHz | Gigahertz |
| MHz | Megahertz |
| PCB | Printed circuit board |
| RF | Radio Frequency |

The present invention provides methods, devices and systems for performing skin treatment by applying to the skin a chemical depilatory composition and heating the skin by application of RF currents. The skin treatment results, inter alia, in efficient hair removal with improved long term depilation.

The terms “depilatory composition” and “depilatory substance” and “depilatory agent” as used herein interchangeably are defined as any substance or compound or mixture of substances or compounds which includes at least one substance or compound that is chemically active in removing hair and/or reducing the rate of hair growth, and/or preventing and/or retarding and/or inhibiting hair growth. However, it is noted that such a depilatory composition or depilatory substance or depilatory agent may also include additional substances and/or compounds including but not limited to, solvents, gelling agents, gels, pH adjusting components, buffer(s), perfume, coloring agents, skin emollients, emulsifiers, skin soothing agents (such as, but not limited to, Aloe Vera extract), inert vehicles and the like, as is known in the art.

The methods and devices of the present invention are based on the application of RF currents to skin which is treated by a depilatory substance (or depilatory composition or agent). The application of RF currents heats the skin tissue and the depilatory substance or composition applied thereto to a temperature higher than the normal skin temperature. This elevation of temperature of the skin and the depilatory possibly increases the rate of the chemical action of the active ingredient(s) of the depilatory substance or composition, accelerates the depilation and improves the immediate as well as the long term effectiveness of the skin treatment.

The superior results obtained by heating the skin using RF currents in the presence of depilatory substance or composition in contact with the skin may be at least partially attributed to the increased RF current density in the tissues regions adjacent the hair shaft and hair follicles resulting in an elevated temperature in these regions and increased efficacy of chemical depilatory action in these regions. This selective effect is obtained by the selection of RF currents for heating the skin.

The details of exemplary (non-limiting) methods and devices used to demonstrate skin treatment using RF currents application to depilatory composition treated skin are listed in the experiments described below.

### Experiment 1

The Depilatory composition selected for use in the experiment was “Veet Rasera—Gel Depilatorio”, moisture enriched depilatory gel, packaged in a spray can, commercially available from RECKITT BENCKISER, UK. The active ingredients indicated by the manufacturer are potassium thioglycolate and calcium hydroxide.

Reference is now made to FIG. 7 which is an isometric view schematically illustrating an exemplary RF current applicator system used for applying RF currents to the skin in the presence of depilatory substances or compositions in accordance with an embodiment of the present invention.

The RF current application system 10 includes a hand-held RF current applicator 11 which was used in the experiments described herein. The system 10 also includes an RF current generating unit 12 rated at 32 Watt total power and 6.4 Watt of power delivered to the skin.

RF currents at a frequency of 1 MHz (sinusoidal AC) and a voltage of 240 Volt (peak to peak) were delivered to the skin through three hemisphere-like electrically conducting RF electrodes 16A, 16B and 16C which where disposed in the RF current applicator 11. The RF currents were delivered as RF bursts having a burst duration of two milliseconds and inter-burst time intervals of eight milliseconds (equivalent to a 20% duty cycle at a 100 Hz burst frequency). The hand-held applicator 11 is suitably connected to the RF current generating unit 12 by suitable isolated electrical conductors (not shown) included in a power cable 13. The RF current generating unit 12 was connected to the mains electrical outlet (not shown) through a regular insulated power cord (not shown for the sake of clarity of illustration). The RF current applicator 11 includes an ON/OFF switch 18 for controlling the application of RF currents to the skin.

It will be appreciated by those skilled in the art that the systems and devices disclosed herein are not limited to being operated by mains alternating currents and may also be operated by any suitable direct current (DC) source, such as but not limited to, a DC power supply, one or more battery, any suitable primary electrochemical cell(s), primary electrochemical cell(s), rechargeable electrochemical cell(s) and fuel cell(s). suitable combinations of power sources may be also used as is known in the art. Such compact power sources may be used in implementing small compact (preferably hand-held systems in which the RF current generating unit may be a small unit built into the RF current applicator 11. Such portable hand-held systems (not shown in FIG. 7) may include any of the above described compact power source(s) within a suitable compartment in the applicator 11.

In the specific applicator used in this experiment, the diameter of each of the RF electrodes 16A, 16B and 16B was five millimeters and the distance between the centers of any two electrodes was ten millimeters. The RF currents were applied to the skin in a bipolar electrode configuration in which one of the electrodes 16A, 16B and 16C was used as a current source and the remaining two electrodes were used the current sink. It is noted that this specific sink/source arrangement is not obligatory and any other suitable electrode configurations may also be used in delivering the RF currents to the skin, depending, inter alia, on the number, type and geometrical arrangement of the electrodes being used.

It is noted that the methods and disclosed herein are not limited to use of the particular RF current applicator 10 disclosed hereinabove. Rather, any suitable type of device capable of delivering RF currents to the skin for heating the skin may be used for implementing the methods of the present invention as will be easily implemented by those skilled in the art, without undue experimentation. For example, the size and shape of the applicator, the number size and configuration of RF electrodes included in the RF device and the parameters of RF currents delivery to the skin may be changed depending, inter alia, on the type of the RF applicator device, the nature of the depilatory composition or substance being used, the dimensions of skin region to be treated and other practical and manufacturing considerations.

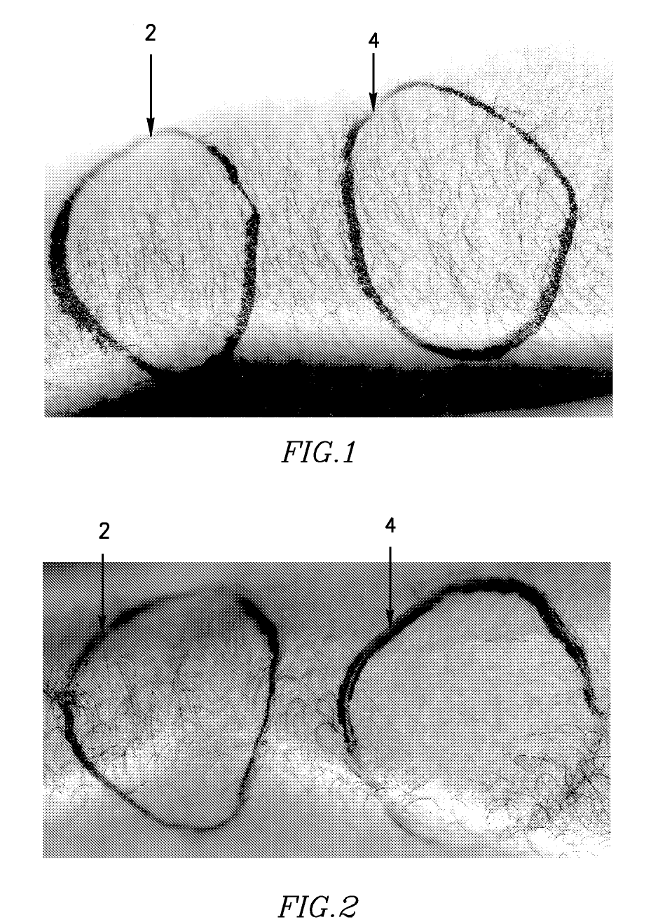

The experiment was performed on the right hand of one of the inventors. Two regions of skin 2 and 4 (of FIGS. 1-2) having a diameter of roughly four centimeters each were marked on the surface of the skin with black paint. The distance between the two marked skin regions 2 and 4 was approximately two centimeters. The two skin regions 2 and 4 were than both sprayed with the VEET—Rasera depilatory gel and a stopwatch was activated to determine the time of treatment.

The skin region 2 was left as it is (for use as a control region) and the region 4 was further treated by applying the RF current applicator 10 to the skin such that the three RF electrodes 16A, 16B and 16C were in contact with the depilatory gel coated skin. The RF current applicator 10 was turned on and moved within the skin region 4 using rotating movements confined within the black paint circle surrounding the skin region 4. the regions 2 and 4 were visually observed during the experiments. At sixty (60) seconds from the start of the RF current application to skin region 4 most of the hairs were observed to be removed from the skin region 4. The RF current application was continued for an additional ten seconds (giving a total time of RF current treatment of seventy seconds) and then the RF current applicator was removed and the skin regions 2 and 4 were both washed with tap water to remove the depilatory gel. The hand was then dried and the part of the hand including the skin regions 2 and 4 was scanned by a model CanoScan LiDE60 scanner commercially available from Cannon, Japan, to record the appearance of the skin regions 2 and 4. The scanner has a resolution of 2400×2400 dpi.

Reference is now made to FIGS. 1-2 which are photographs obtained by the CanoScan scanner illustrating the results of experiments for removing hair by using the combination of a chemical depilatory composition and heating the skin by direct application of RF currents, in accordance with an embodiment of the present invention.

FIG. 1 illustrates the skin regions 2 and 4 before the beginning of the experiment (prior to application of the depilatory gel to the regions 2 and 4). The skin regions 2 and 4 are seen to be covered with hairs.

FIG. 2 illustrates the skin regions 2 and 4 immediately after the termination of the RF current treatment and washing of the treated hand. The control skin region 2 (which was treated with the depilatory gel for about seventy seconds without the application of RF currents) seemed to have a density of hair similar to the density of hairs in the same skin region prior to treatment (compare to the same skin region in FIG. 1). While in the skin region 4 which was treated with the depilatory gel and by application of RF currents thereto, most of the hairs were removed. Upon visual and tactile manual inspection, the skin region 4 was smooth and almost free of hairs. A slight redness of the skin of region 4 was also observed for about five minutes after termination of the RF currents. This redness disappeared after about five minutes.

The results of EXPERIMENT 1 demonstrate that application of RF currents to the skin for a relatively short time period in the presence of a depilatory composition, was effective in removing hairs, while application of the same depilatory composition alone to skin for the same time period was not effective for hair removal.

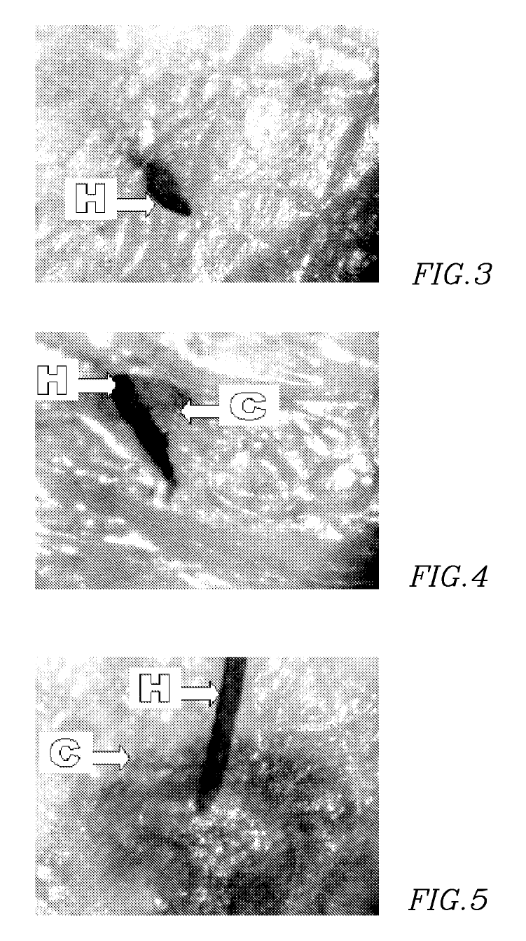

Reference is now made to FIGS. 3 and 4 which are photomicrographs illustrating the skin region surrounding different human hairs after application of a chemical depilatory composition without heating of the skin (FIG. 3) and after combined application of a chemical depilatory composition with RF heating of the skin (FIG. 4), in accordance with an embodiment of the invention. FIGS. 3 and 4 were obtained by enlarging selected parts of scanned images obtained from skin regions 2 and 4 immediately after performing EXPERIMENT 1 on the hand.

In FIG. 3, the stub of a cut hair shaft is marked by a white arrow labeled H. It may be observed that upon application of the depilatory gel without RF current heating the region of the skin region adjacent the hair shaft is substantially unaffected by exposure to the depilatory gel for seventy seconds. In contrast, in FIG. 4 the stub of a (different) cut hair shaft (also marked by a white arrow labeled H in FIG. 4) it may be observed that after application of the depilatory gel and application of RF currents heating for seventy seconds, the region of the skin adjacent the cut hair shaft is substantially damaged as indicated by the formation of a “crater” or depression (indicated by a white arrow labeled C) surrounding the cut hair shaft. In the original color scans (not shown), the crater of damaged tissue had a red color characteristic of follicular erythema.

In later observations of skin regions treated by the combination of the depilatory gel and RF application as described in detail in EXPERIMENT 1 hereinabove it was found that the follicular erythema was present and even more pronounced in appearance.

Reference is now made to FIG. 5 which is a photomicrograph illustrating selective damage of the skin regions surrounding human hairs two days after application of a depilatory composition combined with RF heating of the skin. In FIG. 5 a stub of a cut hair shaft marked by a white arrow labeled H is shown. A crater (the boundary of which is marked by the white arrow marked C may be seen. The crater is larger and more extensive in area and the red region of follicular erythema is spread over a much larger area than the typical craters observed immediately after the treatment (such as the crater C shown in FIG. 4.

It is noted that observations reveal that at times of about 10-15 minutes after treatment the skin regions which are not adjacent to hair shafts were substantially undamaged and showed no signs of inflammation or erythema as indicated by their normal skin color and by the absence of any visual signs of inflammation. It therefore appears that the effect of applying RF currents to depilatory composition treated skin is selectively particularly efficient in the skin region adjacent the hair shaft and/or the hair follicles.

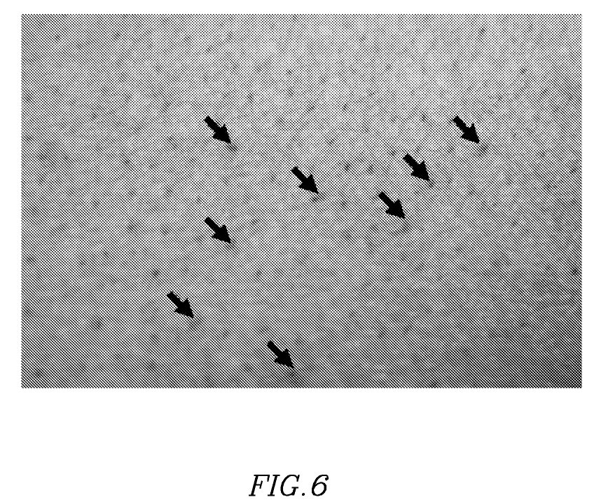

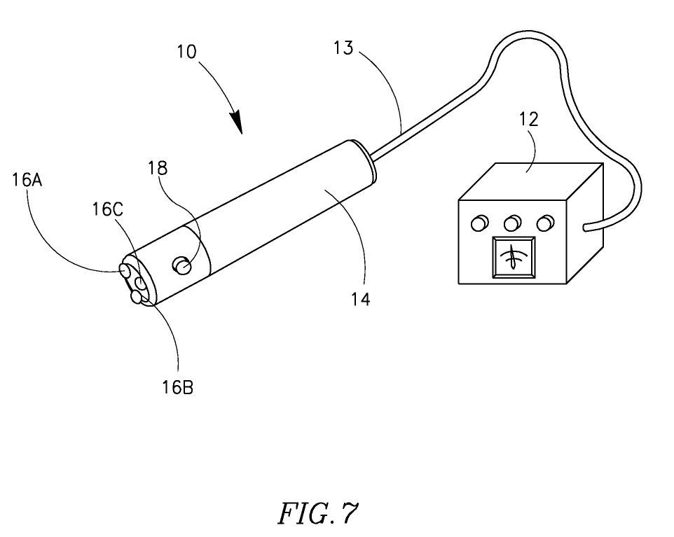

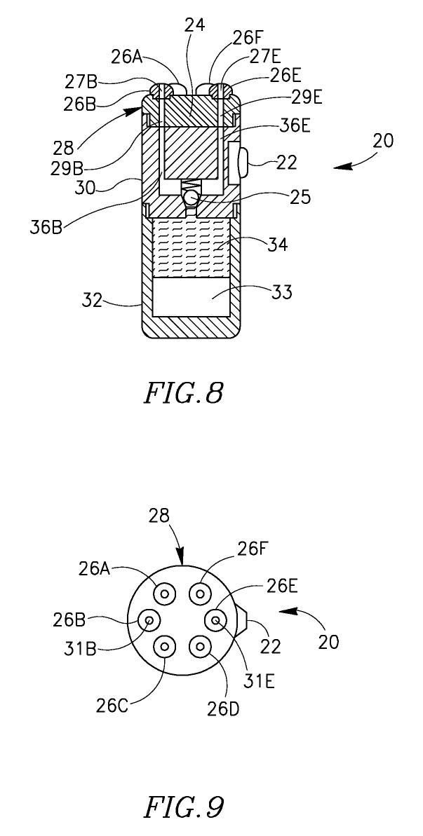

Reference is now made to FIG. 6 which is a photograph illustrating a region of human skin (of the arm) demonstrating localized damage caused to the skin regions surrounding hair shafts after treatment of the skin with a chemical depilatory composition combined with skin heating using the application of RF currents to the skin, in accordance with an embodiment of the present invention. The photograph was taken two days after treating the photographed region of skin with a combination of a depilatory gel and application RF current as disclosed in detail in EXPERIMENT 1 hereinabove. The treated region of the skin was photographed using a Cannon digital camera Model POWERSHOT A620. The original color photographs showed that many of the hair shaft stubs were surrounded by red areas (marked by white arrows in the photograph) indicative of tissue damage and follicular erythema while the areas of skin far from the stubs were substantially devoid of visible signs of erythema or damage.

While the reasons for the selective effect observed are not presently well understood, it is possible that the selectivity may be due to the known higher RF current density in the regions near the hair shaft and hair follicle. This current density distribution is known in the art as published by Kreindel et al. in U.S. Pat. No. 6,889,090 and in U.S. published patent application number 2005/0159737 both of which are incorporated herein by reference in their entirety.

It is therefore possible that due to the higher RF current density near the hair shaft and follicular regions, these areas are selectively preferentially heated by the RF currents to a temperature that is significantly higher than the temperature of skin tissue in the regions distant from the hair shafts or the follicular region (the regions having a lower RF current densities therein). As the rate of many chemical reactions is increased by increasing the temperature (the Q₁₀ value of chemical reactions typically predicts a twofold increase in reaction rate for every 10° C. increase in temperature, as is well known in the art), selective heating of the hair follicle regions may result in the acceleration of keratinolytic reactions and/or other chemical reactions of the active depilatory substance(s) in the depilatory gel or composition with the hair shaft and/or the follicular tissues adjacent the hair shat which may lead to more extensive and/or selective damage in the hair shafts follicular regions as compared to the other skin regions.

It is noted that the parameters of RF current application used in EXPERIMENT 1 are given by way of example only and not intended to limit the parameters of RF current application of the methods and devices disclosed herein. Rather, many of the RF current parameters may be varied, depending, inter alia, on the type of skin to be treated (such as, but not limited to, facial skin, hand skin, leg skin, torso skin and the like), the physical parameters of the RF delivery device being used, the type and/or concentration of depilatory substance(s) being used, and many other considerations.

Thus, while typically the acceptable range of RF energy delivered to the skin may be between 5-25 Watt/cm² of skin (watt per square centimeter of skin), other different values of the delivered RF energy larger or smaller than this range may also be used depending on the application. Similarly, the RF frequency, waveshape (sinusoidal or any other usable waveforms), burst frequency, duty-cycle and the like may be varied depending on the application. For example, while the RF frequency should typically be at least 300 KHz or higher in order to prevent tissue spasms, any other usable RF frequency above spasm-initiating frequencies may be used in the methods and devices described herein. Generally, the RF current parameters and energy applied to the skin may be any combination of parameters sufficient to substantially increase the temperature of the skin (or of specific parts thereof as disclosed hereinabove) so that the chemical depilatory action proceeds faster than on non-RF heated skin, without causing any serious damage to the skin due to extensive skin tissue coagulation or skin burns.

Results of Using Different Commercial Depilatory Compositions

Additional experiments were performed using various different commercially available depilatory compositions. Similar results to the results obtained in EXPERIMENT 1 were obtained when using the RF current applicator 10 and the same experimental design and RF treatment conditions in combination with the following depilatory compositions:

1) “Veet for man” depilatory cream, commercially available from RECKITT BENCKISER UK. (Ingredients: potassium thioglycolate calcium hydroxide).
2) Facial Hair Removing Cream commercially available from BOOTS, UK Ingredient: Potassium Hydroxide
3) “BRUSH-ON FACIAL HAIR REMOVER” commercially available from Nair, UK (Ingredients: sodium hydroxide, calcium hydroxide).
The results obtained using all three of the above commercial depilatory products were very similar to those obtained in EXPERIMENT 1.

### Experiment 2

The experiment was performed for testing the long term efficacy of the novel method disclosed hereinabove of treating skin by the combined application of depilatory substance and RF currents to the skin.

The experiment was performed on both legs of a male test subject having a type III skin (according to the Fitzpatrick scale). The leg hair had brown color.

The depilatory gel used and the RF current application method, were as described in detail in EXPERIMENT 1 hereinabove. Symmetrical regions on the skin of the lower part of both legs (roughly overlying the Soleus muscle) were chosen for treatment. Prior to application of the depilatory gel, the skin regions selected for the experiment were photographed using the CanoScan LiDE60 scanner as described in detail hereinabove (this set of photographs was used for determining the number of hairs before treatment). The two skin regions on the right and left leg of the test subject were covered with the depilatory gel as described hereinabove. The depilatory gel treated skin region on the right leg was further treated by application of RF currents as described in detail for EXPERIMENT 1 hereinabove using the same RF current applicator 10 (of FIG. 7). The duration of application of RF currents was three minutes. The depilatory gel treated skin region on left leg was left undisturbed for three minutes. At the end of RF current application both legs were extensively washed with tap water to remove the depilatory gel from both legs.

Three days after the first treatment, both of the gel treated regions were again photographed using the CanoScan LiDE60 scanner as described in detail hereinabove and the photographs were used for evaluating the reduction in hair numbers at three days after the first depilatory treatment. Fifteen days after the first treatment the same regions were again were photographed using the CanoScan LiDE60 scanner as described in detail hereinabove and the photographs were used for evaluating the reduction in hair numbers at fifteen days after the first depilatory treatment.

After taking the photographs (at 15 days post the first depilatory treatment), the same regions of skin on both the left and the right legs were again subjected to a second depilatory treatment applied as disclosed in detail for the first depilatory treatment. The same left leg skin region was treated with depilatory gel only for three minutes and the same right leg skin region was treated with depilatory gel combined with RF current application for three minutes, as described for the first treatment. After the second treatment was finished, the legs were washed with tap water to remove the depilatory gel. Twenty six days after the first treatment, the same skin regions were again photographed using the CanoScan LiDE60 scanner as described in detail hereinabove and the photographs were used for evaluating the reduction in hair numbers at twenty days after the first depilatory treatment (eleven days after the second depilatory treatment).

The four sets of photographs obtained as described above were used for manually counting the number of hairs in skin areas having the approximate dimensions of 3×3 centimeters on the left and right leg photographs. The results of EXPERIMENT 2 are summarized in TABLE 1 below.

TABLE 1

| | | | | Reduction 26 days | |
|---|---|---|---|---|---|
| Reduction after | | Reduction 15 days | | after first treatment | |
| 3 days after first | | after first treatment | | (11 days after second | |
| treatment (%) | | (%) | | treatment) (%) | |
| R | L | R | L | R | L |
| 50.6 | 21.8 | 34.9 | 1.6 | 21.7 | −1.6 |

The reduction in hair count in the treated skin regions is expressed in percent reduction calculated by dividing the number of hairs counted in the photographs after treatment by the number of hairs counted in the photographs taken before the depilatory treatment and multiplying by 100. The symbols R and L in the table represent the results from the right leg and left leg, respectively. The right leg was the one treated with depilatory gel and RF current application and the left leg was treated by depilatory gel only.

The results in TABLE 1 clearly indicate that in the skin region treated by depilatory gel and RF current application (R) there is a substantial reduction in hair re-growth as compared to the region that was treated by the depilatory gel only (L). Furthermore, with repeated applications of the combined treatment (depilatory gel+RF currents), there was a further unexpected (cumulative) reduction of hair re-growth which was not observed in repeated treatment using depilatory gel only. The reasons for the unexpected observed cumulative reduction of repeated depilatory gel and RF currents applications are not currently understood.

It is noted that while in experiments 1 and 2 described above the depilatory gel was applied manually to the skin prior to the application of RF currents, other devices and systems may be implemented in which the depilatory composition or agent may be contained in or included within the RF applicator device or in the hand applicator of a system as described in detail hereinafter.

Reference is now made to FIGS. 8-9. FIG. 8 is a schematic part cross-sectional diagram illustrating a device having a replaceable RF electrode assembly and a reservoir for applying a depilatory substance or composition to the skin and for applying RF energy to the depilatory substance treated skin, in accordance with an embodiment of the present invention. FIG. 9 is a schematic front view of the device illustrated in FIG. 8.

The device 20 includes a removable RF electrode assembly 28 detachably attached to a handle 30. The removable RF electrode assembly 28 includes six hollow RF electrodes 26A-26F. The RF electrodes 26A-26F are attached to an electrode block 24. The electrode block 24 is an electrode carrying member or electrode carrying plate, preferably but not obligatorily having a disc-like shape (however, any other suitable type of shape may be used for implementing the electrode carrying members of the present application, including but not limited to, plates or discs having ellipsoidal cross-section, rectangular cross-section or any other suitable shape). The RF electrodes 26A-26F have hollow passages formed therein (note that only passages 27B and 27 E of electrodes 26B and 26E, respectively are shown in the cross section of FIG. 8). The hollow RF electrodes 26A-26F are made from an electrically conducting material (such as but not limited to an electrically conducting metal) or may be made from an electrically insulating material (such as, but not limited to, a polymer based material, or plastic) which is coated or plated with an electrically conducting material. The electrode block 24 may be preferably made from an electrically insulating material (such as, but not limited to, a polymer based material, a suitable engineering plastic and the like). The electrode assembly 28 may be attached to the handle 30 by a quick-release mechanism (not shown in detail) or by suitable threads formed therein (not shown), or by any other suitable attachment method or device. The entire electrode block 28 may be detached or removed from the device 20 and may be replaced with a new electrode assembly if the need arises (for example, due to clogging of one or more of the hollow passages in the RF electrodes 26A-26F, or due to corrosion or deterioration of the RF electrodes 26A-26B.).

The device 20 also includes a detachable reservoir 32 detachably attached to the handle 30. When the reservoir 32 is attached to the handle 30, the space 33 within the reservoir 32 may be partially filled with a suitable depilatory substance 34 (such as but not limited to a depilatory gel, a depilatory cream, a depilatory liquid, a depilatory foam, or the like) as described in detail hereinafter. The space 33 may also include a suitable compressed gas or a suitable propellant for pushing or propelling quantities of the depilatory substance 34 through the controllable valve 25 disposed in the handle 30. For example, when the push button 22 attached to the handle 30 is pressed, the valve 25 opens and the propellant (not shown) in the space 33 pushes the depilatory substance through the open valve 25 and into the hollow passages 36B and 36E formed within the handle 30, into the hollow passages 29B and 29E, respectively which are formed within the electrode block 24 and through the corresponding passages 27B and 27E formed within the RF electrodes 26B and 26E, respectively. The depilatory substance 34 finally exits through the openings 31B and 31E in the electrodes 26B and 26E, respectively to be applied to the skin (not shown). It is noted that the corresponding passages in the electrode block and in the handle 30 which are associated with the RF electrodes 26A, 26C, 26D and 26F are not seen in the cross section of FIG. 8.

It is further noted that the electrical conductors connecting the RF electrodes 26A-26F to the RF generating unit (not shown) associated with the device 20 are not shown in FIG. 8 for the sake of clarity of illustration. Such electrical conductors may be implemented as insulated electrically conducting wires disposed in the device 20 or may be implemented as films of electrically conducting material deposited or printed or coated or otherwise attached to or formed on or within the different surfaces or parts of the device 20 (such as, the electrode block 24, the handle 30 and the reservoir 32. Similarly, the RF current generating unit (not shown in FIG. 8 for the sake of clarity of illustration) for supplying the RF currents may be disposed outside of the device 20 (see, for example, the configuration of the system 10 of FIG. 7) or may be a miniature RF current generating unit (not shown in FIG. 8) which may be disposed within the device 20 or a part thereof together with a suitable power source (such as one or more batteries, or the like).

It is noted that preferably (but not obligatorily), the pushbutton 22 when activated may allow the flow of the depilatory substance 34 through the openings in the RF electrodes 26A-26F and onto the skin (not shown) and may also simultaneously initiate the application of RF currents to the skin through the RF electrodes by suitably switching on the RF current generating unit connected to the device 20 or included therein.

It is noted that while in the embodiment of the device 20 (of FIG. 8) each of the RF electrodes 26A-26F has a single hollow passage formed therein, the devices of the present application also includes embodiments in which each (or some) of the RF electrodes 26A-26F has two or more hollow passages (not shown in FIG. 8) or conduits formed therein. This may be advantageous because even if one conduit in an RF electrode becomes clogged during operation of the device the remaining conduits may still function to allow the application of the depilatory substance 34 to the skin.

It is also noted that further embodiments of the device of the present application may be implemented in which only some of the RF electrodes have one or more hollow passage(s) or conduits formed therein while the remaining RF electrodes do not have any passage or conduit formed within them. For example, the device 20 of FIG. 8 may be modified such that only the RF electrodes 26A, 26C and 26E have one or more hollow passages or conduits formed therein (not shown in FIG. 8) such that during operation of the device they may be used for applying the depilatory substance 34 onto the skin and also for delivering RF energy to the skin, while the remaining RF electrodes 26B, 26D and 26F do not have any hollow passage formed therein and are used for applying RF energy to the skin and for mechanically spreading the depilatory substance applied to the skin through the RF electrodes 26A, 26C and 26E.

It will be clear to the person skilled in the art that many variations and combinations of RF electrode structure may be used in the embodiments of the device of the present application and that any possible combinations and geometrical arrangements of RF electrodes selected from RF electrodes with one or more hollow passages formed therein and RF electrodes without any hollow passages formed therein may be used for implementing the skin treating devices of the present application.

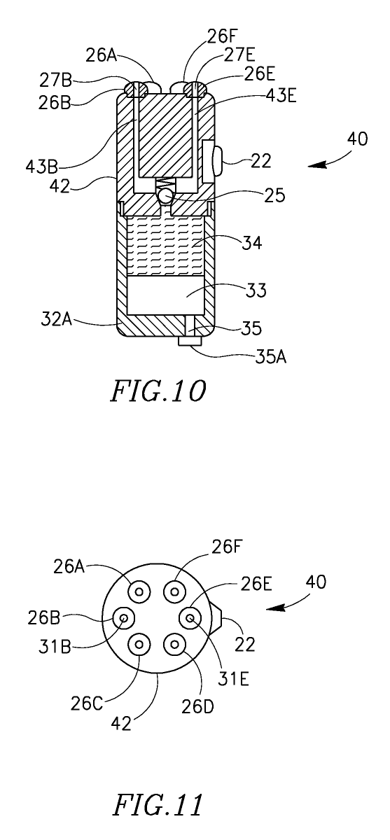

Reference is now made to FIGS. 10-11. FIG. 10 is a schematic part cross-sectional diagram illustrating a device having a refillable reservoir for applying a depilatory substance or composition to the skin and for applying RF energy to the depilatory substance treated skin, in accordance with an embodiment of the present invention. FIG. 11 is a schematic front view of the device illustrated in FIG. 10.

The device 40 includes an RF electrode assembly 42 detachably attached to a refillable reservoir 32A. The RF electrode assembly 42 includes six hollow RF electrodes 26A-26F. The RF electrodes 26A-26F are suitably attached to the electrode assembly 42. The RF electrodes 26A-26F have hollow passages passing formed therein (note that only passages 27B and 27 E of electrodes 26B and 26E, respectively are shown in the cross section of FIG. 10). The hollow RF electrodes 26A-26F are constructed as described in detail hereinabove. The electrode assembly 42 may be preferably (but not obligatorily) made from an electrically insulating material (such as, but not limited to, a polymer based material, or plastic or the like), However, metal, or other suitable structural materials may also be used for forming the Electrode assembly 42. The electrode assembly 42 may be attached to a refillable reservoir 32A by a quick-release mechanism (not shown in detail) or by suitable threads formed therein (not shown), or by any other suitable attachment method or device.

The space 33 within the refillable reservoir 32A may be partially filled with a suitable depilatory substance 34 (such as but not limited to a depilatory gel, a depilatory cream, a depilatory liquid, a depilatory foam, or the like) as described in detail hereinafter. The space 33 may also include a suitable compressed gas or a suitable propellant for pushing or propelling quantities of the depilatory substance 34 through the controllable valve 25 disposed in the electrode assembly 42. For example, when the push button 22 attached to the electrode assembly 42 is pressed, the valve 25 opens and the propellant (not shown) in the space 33 pushes the depilatory substance through the open valve 25 and into the hollow passages 43B and 43E formed within the electrode assembly 42, into the corresponding hollow passages 27B and 27E formed within the RF electrodes 26B and 26E, respectively. The depilatory substance 34 finally exits through the openings 31B and 31E in the electrodes 26B and 26E, respectively to be applied to the skin (not shown). It is noted that the corresponding passages in the RF electrode assembly 42 which are associated with the RF electrodes 26A, 26C, 26D and 26F are not seen in the cross section of FIG. 10.

The reservoir 32A has a filling passage 35 formed therein. A one way valve 35A seals the filling passage 35. The reservoir 32A may be filled (or refilled) with depilatory substance 34 and/or with a suitable propellant or compressed gas, through the valve 35A as is well known in the art. Thus, when the depilatory substance 34 runs out, the reservoir 32A may be refilled from a re-supply canister or container (not shown) containing the depilatory substance and a propellant or pressurized gas through the valve 35A as is well known in the art (for example, in a way similar to the way used for refilling an empty refillable lighter with liquefied butane/propane mixture).

It is further noted that the electrical conductors connecting the RF electrodes 26A-26F to the RF generating unit (not shown) associated with the device 40 are not shown in FIG. 10 for the sake of clarity of illustration. Such electrical conductors may be implemented as insulated electrically conducting wires disposed in the device 40 or may be implemented as thin films of electrically conducting material (such as, for example, gold or another metal) deposited or printed or coated or otherwise attached to or formed on or within the different surfaces or parts of the device 40 (such as, the electrode assembly 42, and the reservoir 32). Similarly, the RF current generating unit (not shown in FIG. 10 for the sake of clarity of illustration) for supplying the RF currents may be disposed outside of the device 40 (see, for example, the configuration of the system 10 of FIG. 7) or may be a miniature RF current generating unit (not shown in FIG. 8) which may be disposed within the device 40 or a part thereof together with a suitable power source (such as one or more batteries, or the like).

It is noted that preferably (but not obligatorily), the pushbutton 22 when activated may allow the flow of the depilatory substance 34 from the reservoir 32A through the openings in the RF electrodes 26A-26F and onto the skin (not shown) and may also simultaneously initiate the application of RF currents to the skin through the RF electrodes by suitably switching on the RF current generating unit (not shown in FIG. 10) or by electrically connecting the some or all of the RF electrodes 26A-26F to the RF current generating unit.

It is noted that the detachable reservoirs 32 and 32A (of FIGS. 8 and 10, respectively) may be detached from the devices 20 and 40, respectively, for cleaning and/or for refilling with the depilatory substance 34. The reservoir 32A may also be detached for filling with the depilatory substance 34 and then reattached to the RF electrode assembly 42 after which a suitable propellant or compressed gas may be introduced into the space 33 through the valve 35A.

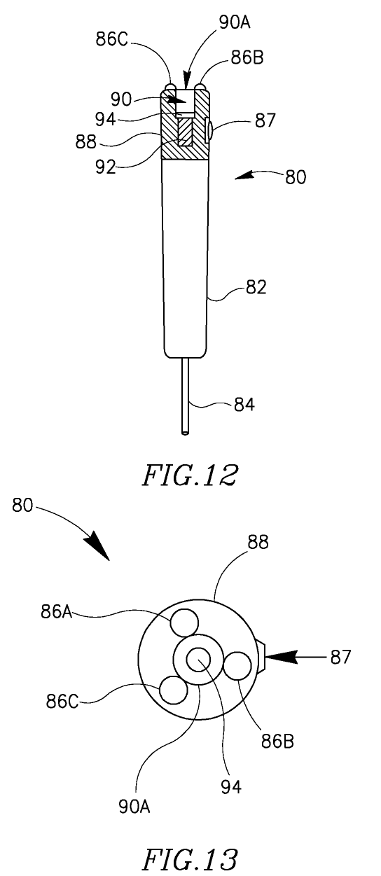

Reference is now made to FIGS. 12-13. FIG. 12 is a schematic part cross-sectional diagram illustrating a device for applying RF energy to skin treated with a depilatory substance. The device has an infrared thermometer for monitoring skin temperature in accordance with an embodiment of the present invention. FIG. 13 is a schematic front view of the device illustrated in FIG. 12.

The hand held RF current applicator 80 may form part of a desktop system (note that only the hand-held applicator 80 is shown in FIG. 12). The applicator 80 includes a housing 82 and an RF power cable 84 attached thereto. The power cable 84 may be attached to an RF current generator unit (not shown in FIG. 12) such as, but not limited to the RF current generating unit 12 of FIG. 7.

The housing 82 also includes an electrode assembly 88 with three RF electrodes 86A, 86B and 86C. The housing 82 also includes an activating switch 87 attached thereto for starting and stopping the application of RF currents to the skin. The electrode assembly 88 has an open recess 90 formed therein. An infra-red (IR) optical thermometer unit 92 is disposed in the electrode assembly 88 with the IR sensor unit 94 of the IR thermometer unit 92 facing the recess 90. When the applicator 80 is placed on the skin, the IR thermometer unit 90 may measure the temperature of the skin region adjacent the opening 90A of the recess 90 by sensing the IR radiation emanating from the skin and/or from any depilatory substance disposed on the skin underlying the opening 90A. The results of the temperature measurement may be used by the system including the applicator 80 to monitor the skin temperature. For example, if the skin temperature as determined by the IR thermometer unit 90 exceeds a certain threshold value, the application of RF currents to the skin may be automatically stopped in order to avoid a skin burn or undesirable coagulation or damage of skin tissues. The threshold value may be predetermined or preset by the manufacturer of the system or the device.

It will be appreciated by those skilled in the art that other embodiments including variations of the design and construction of RF current applicators capable of dispensing a depilatory agent onto the skin surface may be implemented.

For example, the depilatory substance 34 (and, if necessary the propellant) may be supplied to the skin by using a suitable pressurized disposable canister or cartridge (not shown) which may be placed for example in the space 33 of the reservoir 32 (FIG. 8). The canister may have a valve similar to the valve 25 which may function in association with the pushbutton 22 in a similar way to the valve 25. An advantage of replacing the fixed valve 25 with a similar valve built into the canister is that there is no need to clean the valve. Such a depilatory substance containing canister is preferably disposable and when the supply of the depilatory substance in the canister is exhausted, the entire canister (including the valve) may be disposed and replaced with another new canister.

It may also be possible to implement the devices described herein by using a suitable pump (not shown) included in the devices 20 and/or 40. For example, the pump may be a small electrical fluid pump preferably replacing the valve 25 (of FIGS. 8 and 10). The pump may be activated by the pushbutton 22 to pump the depilatory substance 34 from the reservoirs 32 or 32A into the RF electrodes and onto the skin. In embodiments including such a pump, the space 33 of the reservoirs 32 and/or 32A need not contain a propellant or compressed gas, as the pump will actively pump the fluid substance 34. In such embodiments including a pump, the reservoir 32 (FIG. 8) may be recharged with a depilatory substance 34 by detaching, refilling and reattaching the reservoir 32 to the handle 30 and the reservoir 32A (FIG. 10) may be recharged with a depilatory substance 34 by refilling the reservoir 32A through the valve 35A as described hereinabove.

It is noted that for systems including a hand held RF current applicator connected to a separate RF current generator unit (such as but not limited to the system 10 of FIG. 7), the entire RF current hand applicator may be made as a disposable unit. For example, in a system in which one of the devices 20 or 40 (of FIGS. 8 and 10, respectively) are configured as hand applicators attachable to a separate RF current generating unit (such as but not limited to the RF current generating unit 12 of FIG. 7), the device 20 and or the device 40 may be disposable devices which are designed to be disposed off after a single use or after a certain number of uses. Since the expensive RF current generating unit of such embodiments will not included in the device 20 or 40, it will be economical to dispose of the devices 20 and or 40 after a single use or after a certain number of uses. The implementation of such disposable hand applicators may be advantageous in cases in which multiple patients are to be treated by a system operated by a cosmetician or another user. Thus, the use of a disposable hand applicator for a single patient may reduce the risk of transferring an infecting organism (such as, for example, fungi, fungal spores, and bacteria) from one patient to another patient. Alternatively, the user may reserve a multiple use detachable RF hand applicator for use on a single patient. When another patient is to be treated, the hand applicator is detached from the RF current generating unit of the system and another different RF hand applicator is connected to the RF current generating unit for treating another patient.

In such an embodiment of the system, the replaceable hand applicator (such as the device 20 or 40) may be easily attached and detached from the RF current generating unit of the system (such as, but not limited to the RF current generating unit 12 of FIG. 7) by including a suitable electrical connector socket in the device 20 and/or 40 and by modifying the power cable 13 to include a suitable electrical connector (not shown) at the end of the cable 13 which is to be attached and detached to the device 20 or 40 or to any other suitable type of connectable RF current hand applicator.

It will be appreciated by those skilled in the art that the passages passing through the RF electrodes as described hereinabove for applying the depilatory composition to the skin are not obligatory and the devices shown above may be modified for applying the depilatory composition or substance through other parts of the RF applying device.

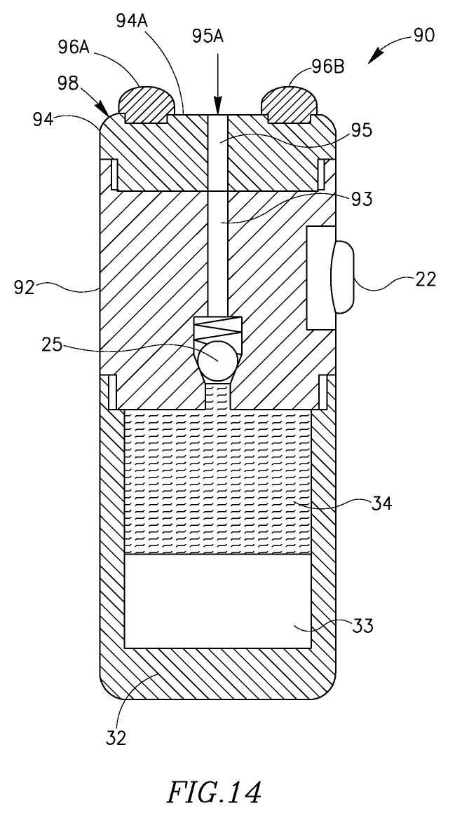

Reference is now made to FIG. 14 which is a schematic part cross-sectional diagram illustrating a device having a replaceable RF electrode assembly having a passage passing therethrough and a reservoir for applying a depilatory substance or composition to the skin and for applying RF energy to the depilatory substance treated skin. In the device 90, the reservoir 32 is constructed as described for the device 20 of FIG. 8 and attached to a handle 92, the space 33 and the depilatory substance 34 is disposed in the reservoir 32 and propelled by a propellant or compressed gas disposed in the space 33 as described for the device 20 hereinabove. The handle 92 has a hollow passage 93 formed therethrough. A controllable valve 25 is disposed in passage 93 for controlling the flow of the depilatory substance 34 into the passage 93. The valve 25 is controlled by the pushbutton 22 as described for the devices 20 and 40 hereinabove. A detachable RF electrode assembly 98 is detachably attached to the handle 92. The RF electrode assembly 98 includes an electrode block 94 and a plurality of RF electrodes (including but not limited to the RF electrodes 96A and 96B shown in the cross sectional view of FIG. 14)) attached to the electrode block 94. It is noted that while only two RF electrodes 96A and 96B are shown in the cross-sectional view of FIG. 14, the device 90 may have any desired number of RF electrodes (typically, two or more electrodes). The electrode block 94 has a hollow passage 95 formed therein.

The passage 95 has an opening 95A at the surface 94A of the Electrode block 94. When the RF Electrode assembly 98 is attached to the handle 92, the hollow passage 95 is contiguous with the hollow passage 93. In operation, when the pushbutton 22 is pressed, the valve 25 is opened and the depilatory substance 34 is pushed by the propellant or compressed gas disposed in the space 33. A quantity of the depilatory substance 34 may then flow through the valve 25, the passage 93 and the passage 95, and exit from the opening 95A to be deposited on the skin (not shown). The pressing of the pushbutton 22 may also initiate the application of RF currents to the skin by the RF electrodes (including but not limited to the RF electrodes 96A and 96B of FIG. 14).

It will be appreciated by those skilled in the art that the specific methods and configurations for applying a depilatory composition illustrated disclosed hereinabove and illustrated in the drawings are not intended as limiting the design of the RF energy application device. Many variations and modifications for applying a flowable substance from a reservoir to the skin are possible as is known in the art, all of which may be implemented by those skilled in the art without undue experimentation, and are intended to be included within the scope of the invention. For example, while the device 90 of FIG. 14 has only one passage 93 formed within the handle 92, only one passage 95 formed within the electrode block 94 and a single opening 95A opening on the surface 94A of the electrode block 94, this is not obligatory and any desired number and configuration of such passages and openings may be used in implementing of the device 90 (or any other of the devices disclosed in the present application). For example, any desired number of passages (with or without associate valve(s)) may be formed within the handle 92, and any number of passages and openings thereof may be formed in the electrode block 24 of the device 90. Such exemplary embodiments may include but are not limited to, an embodiment having three (or any other number) passages formed in the handle 92 and three passages formed in the electrode block 24 and opening in three corresponding openings on the surface 94A.

In another possible embodiment, the handle 92 may have a single valve 25 and a single passage 93 formed therein (as shown in FIG. 8) while the electrode block 94 has three hollow passages (not shown in FIG. 8) obliquely arranged in the electrode block 94 such that they all open opposite the single passage 93 disposed in the end of the electrode block 94 facing the handle 92 while their other openings on the surface 94A of the electrode block 94 are disposed at different separate positions on the surface 94A. Such embodiments may have the benefit that if one or more of the multiple openings or the multiple passages formed in the electrode block 24 is clogged, the depilatory substance 34 may still be applied to the skin through the remaining passages and openings that are not clogged.

It is noted that while the devices illustrated in FIGS. 7-14 have fixed RF electrodes and the devices are manually moved on the surface of the skin to assist in the spreading of the depilatory substance and to obtain amore even distribution of the RF energy in the skin, this is not obligatory and other embodiments of the device may be implemented using movable electrodes and movable electrode assemblies.

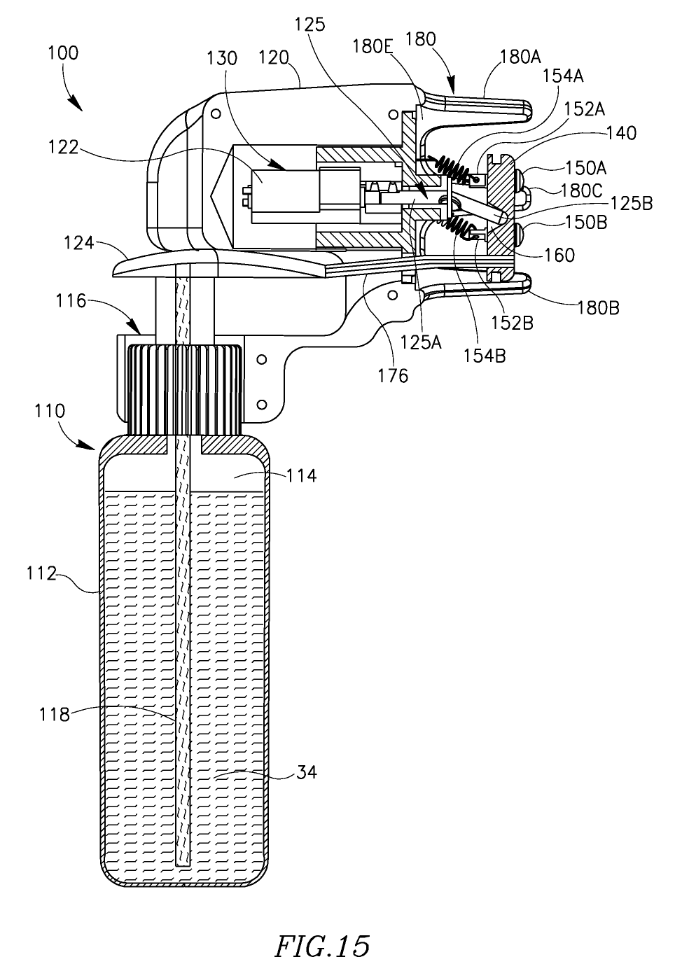

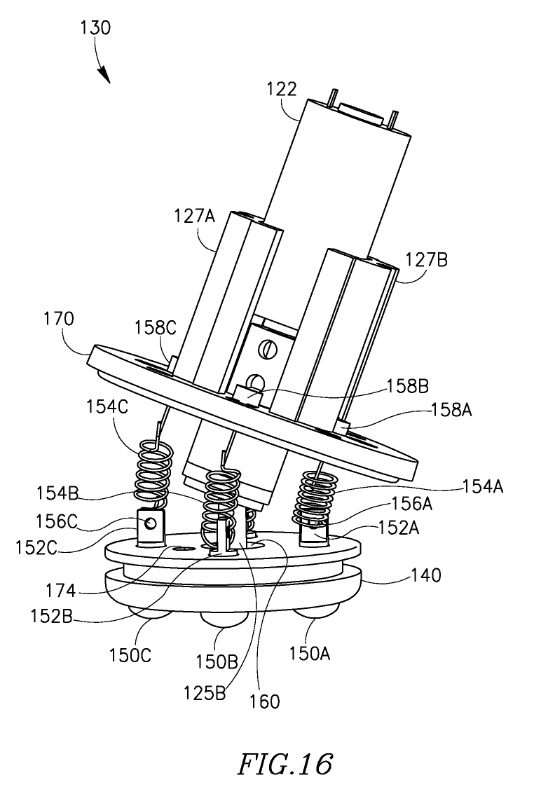

Reference is now made to FIGS. 15 and 16. FIG. 15 is a schematic part cross-sectional part isometric side view illustrating a device having a hand operated pumping dispenser for dispensing depilatory substance and a motorized movable RF electrode assembly, in accordance with an embodiment of the skin treating devices of the present application. FIG. 16 is a schematic side view illustrating the motorized RF electrode assembly of the device of FIG. 15.

The device 100 includes a dispensing unit 110 and a housing 120 attached to the dispensing unit 100. An RF electrode assembly 130 is attached to the housing 120. The RF electrode assembly includes a motor 122. The motor 122 is preferably an electrical motor, but any other suitable types of motors may also be used in implementing the device 100. The motor 122 receives electrical power from a suitable power supply (not shown in FIG. 15) through suitable electrically conducting wires (not shown in FIG. 15 for the sake of clarity of illustration) The motor 122 has a rotatable shaft 125. The shaft 125 has a first shaft portion 125A and a second shaft portion 125B. The first shaft portion is proximal to the motor 122. The second shaft portion 125B is distal to the motor and is bent at an angle to the first shaft portion 125A. When the motor 122 is operating, the shaft 125 rotates and the end of the second shaft portion together with an RF electrode carrying member 140 movably attached thereto rotate in a circular pattern.

The RF electrode carrying member 140 is formed as a generally disc-like plate or block, preferably made of an electrically insulating material (such as but not limited to, a suitable polymer based material, an engineering plastic, polycarbonate, polypropylene, and the like). Electrically conducting RF electrodes 150A-150D are attached to the RF electrode carrying member 140 (It is noted that only RF electrodes 150A and 150 B are shown in the part cross sectional view of FIG. 15 for the sake of clarity of illustration, however, see the isometric view of FIG. 16 for a view of all the RF electrodes 150A-150D). The RF electrode carrying member 140 has a (preferably conic) depression 160 formed therein and the second portion 125B of the motor shaft 125 is movably disposed within the depression 160. When the shaft 125 rotates, the RF electrode carrying member 140 is rotatably moved by the second portion 125B engaged in the depression 160, while allowing the RF electrode carrying member 140 to move at an angle with respect to the second portion 125B, allowing the RF electrode carrying member 140 to follow and accommodate the contours of the treated skin (not shown).

The RF electrodes 150A-150D may be any suitable type of RF electrodes as described hereinabove and as is known in the art. The RF electrodes 150A-150D are electrically connected to four electrically conducting springs 154A-154B, respectively, through electrically conducting holding tabs 152A-152D, respectively. Each RF electrode of the RF electrodes 150A-150D is thus electrically coupled to a different spring of the springs 154A-154D through one of the holding tabs 152A-152D. The holding tabs 152A-152D are preferably made from an electrically conducting material, such as a metal or any other strong electrically conducting material and each tab is electrically coupled to a different RF electrode of the RF electrodes 150A-150D. The springs 154A-154D are preferably made of an electrically conducting metal (such as but not limited to stainless steel and the like). The tabs 152A-152D have holes 156A-156D, respectively, formed therein for attaching the springs 150A-150D, respectively (it is noted that only the holes 156C and 156A are seen in the isometric side view of FIG. 16).

A first end of each spring of the springs 150A-150D is movably attached to one tab of the tabs 152A-152D, respectively, through the hole formed in each tab. The second end of each of the springs 150A-150D passes through a base plate 170 of the motorized RF electrode assembly 130 (to which the motor 122 is attached by motor supporting members 127A and 127B) and is electrically connected to a corresponding electrical connector of four electrical connectors 158A-158B, respectively (it is noted that only the electrical connectors 158A, 158B and 158C out of the four electrical connectors 158A-158D are shown in the isometric side view of FIG. 16). The RF electrodes 150A-150D are suitably electrically connected to an RF energy source (not shown in FIGS. 15-16) by using suitable electrically conducting wires (not shown) electrically connected from the RF energy source to the electrical connectors 158A-158D (it is noted that the electrically conducting wires connecting the RF energy source to the electrical connectors 158A-158D are not shown in FIGS. 15-16 for the sake of clarity of illustration).

The four springs 150A-150D hold the RF electrode carrying member 140 under tension for stabilizing it and allow the RF electrode carrying member 140 to flexibly move in the directions represented by the double headed arrows 172A and 172B for following the contours of the treated skin during operation.

The RF electrode carrying member 140 has a hollow passage 174 passing therethrough. A first end of a flexible depilatory substance supplying conduit 176 (such as, for example, a flexible hollow tube) is disposed within the passage 174 to allow the dispensing of the depilatory substance 34 from the dispensing unit 110 onto the skin. The other end of the conduit 176 is fluidically coupled to the dispensing unit 110 to allow the flow of the depilatory substance 34 from the dispensing unit 110 through the conduit 176 disposed in the passage 174 so that the depilatory substance 34 may exit from the opening of the conduit 176 and reach the surface of the treated skin. When the motor 122 is operating, the rotatory movement of the RF electrode carrying member 140 assists in spreading the depilatory substance 34 extruded from the conduit 176 on the treated skin surface

The dispensing unit 110 includes a manual pumping mechanism 116 and a reservoir 112 detachably attached to the pumping mechanism 116. The reservoir 112 has a space 114 therein which may be filled with the depilatory substance 34. A tube 118 is fluidically connected to the pumping mechanism 116 and extends into the reservoir 112. The pumping mechanism 116 includes a pumping handle 124. The conduit 176 is fluidically sealingly attached to an opening disposed at the end of the pumping member 124.

When the pumping handle 124 is manually activated (by pushing the pumping handle 124) by the user of the device 100, the depilatory substance is pumped through the tube 118 and passes through the pump 116 and the conduit 176 and is dispensed onto the skin through the open end of the conduit 176 distal from the pump 116.

It is noted that the structure and operation of hand operated pumped substance dispensers are well known in the art, are not the subject matter of the present application and are therefore not shown in detail hereinafter. For example, many types of such dispensers are commercially available for dispensing liquid soap, cosmetic creams, perfumes and the like.

The device 100 also includes a stabilizing member 180 detachably attached to the housing 120. The stabilizing member 180 is a crown-like member, preferably (but not obligatorily) made from plastic or a suitable polymer based material (such as, but not limited to polycarbonate, polypropylene and the like). The stabilizing member has a first portion 180E detachably attached to the housing 120. The stabilizing member also has a second portion comprising four spaced apart skin contacting members 180A-180D (It is noted that only three of the skin contacting members 180A, 180B and 180D are seen in the cross-sectional view of FIG. 15).

In operation, the device 100 is pressed against the skin such that at least some (and, preferably, all) of the skin contacting members 180A-180D are in firm contact with the skin. The user than activates the motor 122 to rotate the RF electrode carrying member 140 as disclosed hereinabove. The skin contacting members 180A-180D of the stabilizing member 180 stabilize the position of the device 100 on the skin by preventing or reducing any movements which, in the absence of the stabilizing member 180, may have been caused by the rotational movements of the RF electrode carrying member 140 pushes against the skin. When the stabilizing member 180 is attached to the device 100 such movements are advantageously prevented or significantly reduced.

It is noted that while the stabilizing member 180 of the device 100 is attached to the housing 120, the device 100 may also be modified such that the stabilizing member 180 is attached to the RF electrode assembly 130.

It is further noted that while the stabilizing member 180 is implemented as a crown-like structure having four spaced apart skin contacting members 180A-180D, other embodiments of the device may be implemented in which the shape, size, and number of skin contacting members may be modified. For example stabilizing members having two, three, five, six or any number greater than six of skin contacting members may also be used.

The stabilizing member 180 may be detached from the housing 120 for cleaning purposes. Additionally, removing the stabilizing member 180 may be useful for treating the skin of certain body regions, such as but not limited to the “bikini line” area in the crotch region, the arm pits and the like, which may be more amenable to treatment with the stabilizing member 180 removed from the device 100.

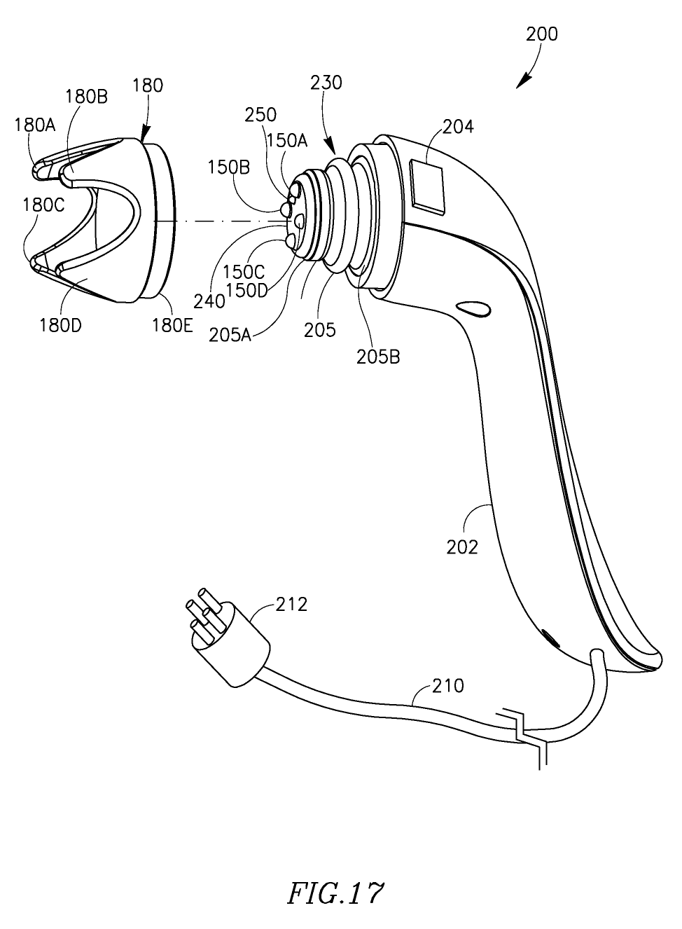

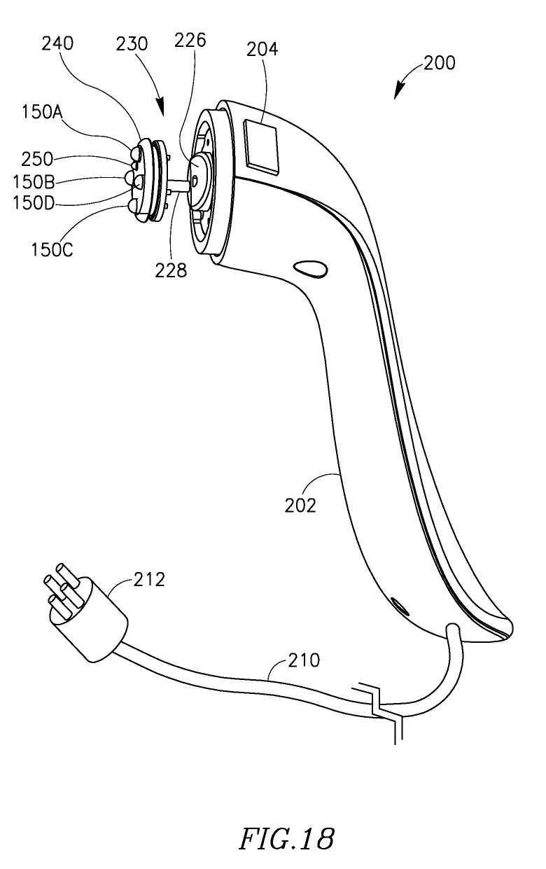

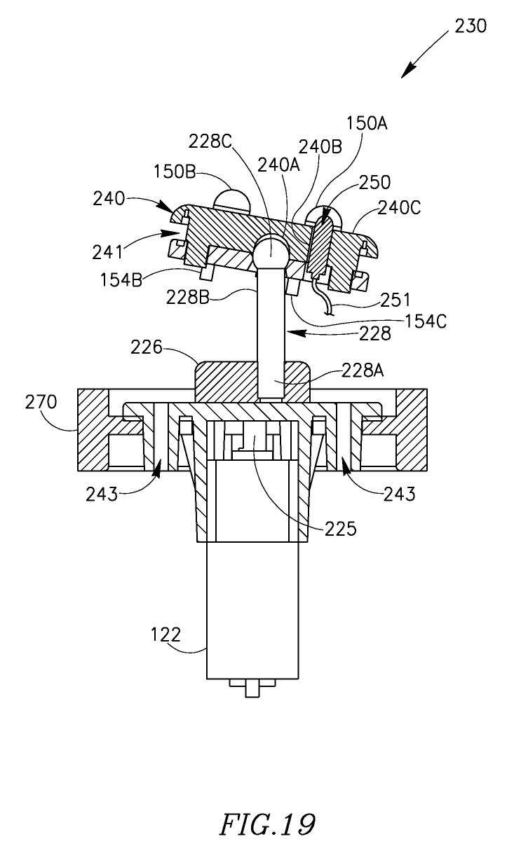

Reference is now made to FIGS. 17-19. FIG. 17 is a schematic exploded isometric view of a skin treatment device having a stabilizing member and a flexible RF electrode assembly protecting sleeve member, in accordance with an embodiment of the skin treating devices of the present application. FIG. 18 is a schematic isometric view illustrating the skin treatment device of FIG. 18 without the stabilizing member and the flexible electrode assembly protecting sleeve. FIG. 19 is a schematic cross-sectional diagram illustrating in detail the motorized movable RF electrode assembly of the skin treatment device of FIG. 18.

The device 200 is a hand held device having a housing 202. The housing 202 may be made from a plastic material or polymer based material such as but not limited to polycarbonate, polypropylene and the like but other suitable materials may also be used. The device 200 also includes the stabilizing member 180 as described in detail hereinabove. The stabilizing member 180 has a first portion 180E which is detachably attached to the housing 202 of the device 200 (the Stabilizing member 180 is shown as detached from the housing 202 in the exploded view of FIG. 17) The stabilizing member 180 also has a second portion comprising four space apart skin contacting members 180A-180D as disclosed hereinabove. The device 200 also includes a movable motorized RF electrode assembly 230 attached to the housing 202.

Turning to FIG. 19, the RF electrode assembly 230 includes a base plate 270 to which a motor 122 is attached. The motor has a rotating shaft 225. A coupling member 226 is attached to the shaft 225. A second shaft 228 is attached at one shaft end 228A to the coupling member 226 and at another shaft end 228B to an RF electrode carrying member 240. The RF electrode carrying member 240 has a generally spherically shaped socket 240A formed therein. The second end 228B of the second shaft 228 has a generally spherically shaped member 228C formed at its end. The spherically shaped member 228C is movably disposed within the socket 240A to form a “ball and socket” joint allowing the RF electrode carrying member 240 to move at various different angles to the longitudinal axis of the shaft 228 to allow the RF electrode carrying member 240 to accommodate for the contours of the skin during operation of the device 200.

The RF electrode carrying member 240 includes four RF electrodes 150A-150D attached thereto. The RF electrodes 150A-150D are constructed and operative as disclosed hereinabove for the device 100 (of FIG. 15-16). It is noted that suitable insulated electrically conducting wires (not shown in FIGS. 17-19 for the sake of clarity of illustration) are connected to the RF electrodes 150A-150D through four electrical connectors 154A-154D (it is noted that only the electrical connectors 154B and 154C are shown in the cross-sectional view of FIG. 19) for energizing the RF electrodes 150A-150D.

The RF electrode carrying member 240 has a hollow passage 240B passing therethrough. A temperature sensor 250 is attached within the passage 240B and protrudes beyond the surface 240C of the RF electrode carrying member 240. In operation of the device 200, the temperature sensor 250 contacts the skin and/or the depilatory substance 34 applied to the skin and senses the temperature of the skin and/or the depilatory substance 34. Preferably, the sensor 250 is a small thermistor having a low thermal mass. However, any other suitable type of temperature sensor known in the art may also be used for implementing the temperature sensor 250. The output signal of the sensor 250 is carried by a sensor wire 251 (only part of the sensor wire is shown in FIG. 19, for the sake of clarity of illustration). The sensor wire 251 as well as the electrically conducting wires for energizing the RF electrodes (not Shown) pass through open passages 243 formed in the base plate 270, and may exit the housing 202 through a suitable cable 210 ending in a suitable electrical connector 212. The Electrical connector 212 may be used to connect the device 200 to an external RF power source (not shown in FIGS. 17-19 but may be similar to the RF current generating unit 12 of FIG. 7 modified to include a DC power supply) and/or to a suitable power source (not shown) for energizing the motor 122 (such as, but not limited to, a DC power source).

The RF electrode assembly 230 includes a flexible sleeve-like member 205 having a first end 205A and a second end 205B (see FIG. 17). The sleeve-like member 205 may be made of any suitable elastic or flexible material, such as but not limited to rubber, synthetic rubber, a flexible or elastic polymer based material and the like, but any other suitable materials may also be used. The sleeve-like member 205 is preferable shaped to have a folded, bellows-like shape or a concertina-like shape or an accordion-like shape to increase it's flexibility.

The RF electrode carrying member 240 has a circumferential slot 241 formed therein for attaching the first end 205 of the sleeve-like member 205 therein. The base plate 270 has a circular slot 271 formed therein for attaching the second end 205B of the sleeve-like member 205 therein.

Preferably, the ends 205A and 205B of the sleeve-like member 205 are sealingly attached to the slots 241 and 271, respectively. The sleeve-like member 205 functions to flexibly stabilize the RF electrode carrying member 240 such that when the RF electrode carrying member 240 is rotated at an angle to the shaft 228 while following the contours of the skin, the force exerted by the flexible (elastic) sleeve like member 205 will act as a returning force which will re-align (straighten) the RF electrode carrying member 240 once the device is lifted off the skin surface. Another function of the sleeve-like member 205 is protecting the inner components of the RF electrode assembly 240 from being contaminated and clogged by the depilatory substance 34 dispensed on the skin.

In operation, the user applies a depilatory substance or a depilatory composition, such as a depilatory cream to the skin. The application may be made manually (by the fingers) or by using any suitable applicator and/or container and/or dispensing device known in the art and containing the depilatory substance or composition. After the depilatory composition has been applied to the skin, the device 200 may be pressed against the skin such that the RF electrodes 150A-150D are in contact with the depilatory treated surface of the skin. The user may then press the activator button 204 on the device 200 to energize the motor 122 for starting the rotating action of the RF electrode carrying member 240 and for energizing the RF electrodes 150A-150D. The rotating action of the RF electrode carrying member 240 assists in spreading the depilatory substance on the treated skin region and the application of the RF energy to the depilatory substance treated skin heats the skin and the depilatory substance to achieve a highly efficient depilation of skin hair as described in detail in the various different experiment disclosed in the present application.

It is noted that while the temperature sensor 250 (of FIG. 19) is disposed in the device 200 to sense the temperature of the skin (and/or of the depilatory substance adhering to the skin), it is possible in accordance with another embodiment of the devices described in the present application to include in the device one or more temperature sensors attached to or disposed within one or more of the RF electrodes of the device (such as, but not limited to, the RF electrodes 150A-150D of the device 200). It may also be possible to use a combination of temperature sensors for determining both the temperature of one or more RF electrodes and the temperature of the skin (and/or the depilatory substance applied to the skin).

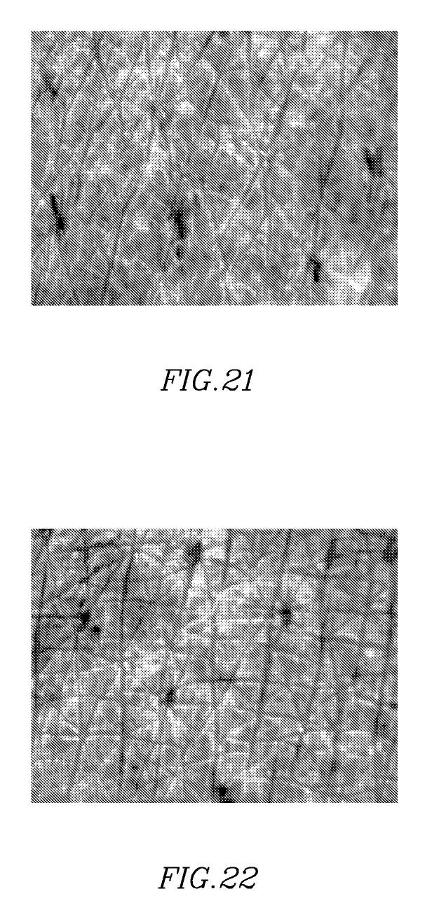

Reference is now made to FIG. 21 is a schematic cross-sectional diagram illustrating part of a skin treating device having a disposable depilatory substance reservoir, in accordance with an embodiment of the skin treating devices of the present application.

The device 300 includes a openable housing 302, a motorized RF electrode assembly 330 and a depilatory substance dispensing unit 310. The housing 302 has a housing body 302A and an openable cover 302B. The openable cover 302B includes a pressure exerting mechanism 320 for exerting pressure on a squeezable elastic reservoir 328 disposed in the housing body 302A. The pressure exerting mechanism 320 includes a compartment 315 formed within the housing body 302A and defined by a printed circuit board (PCB) 322 disposed in the housing body 302A and a pressure plate 324 which is spring loaded by a leaf spring 326. The leaf spring 326 and the pressure plate 324 are movable attached within the openable cover 302B. The printed circuit board 322 may include various electronic components for energizing the RF electrodes of the RF electrode assembly 330, for controlling the operation of the motorized RF electrode assembly and for receiving and/or processing signals from any temperature sensors included in the device 300.

The cover 302B is openably attached to the housing body 302A by a hinge 303 such that it may be opened and closed by moving it in the directions represented by the double headed arrow 301. After closing the cover 302B, the cover 302B may be locked in place by a suitable openable latch or clamping mechanism (the latch is not shown in FIG. 21 for the sake of clarity of illustration).

The depilatory substance dispensing unit 310 includes a squeezable (elastic) reservoir 328 including a depilatory substance 34 therein. The reservoir 328 is preferably an elongated pouch (or bag, or sachet) made from a strong thin pliable material (such as, but not limited to polyethylene or any other suitable plastic or polymer based material). However, other suitable pliable or flexible materials known in the art may also be used in making the squeezable reservoir 328. The dispensing unit 310 includes a hollow flexible dispensing tube 328A attached to the reservoir 328 or formed as a contiguous part thereof.

The RF electrode assembly 330 includes an electrical motor 122 attached to a base plate 370. A coupling member 326 is attached to the shaft 327 of the motor 122 and a second shaft 328 is eccentrically attached to the coupling member 326. The second shaft 328 and is movably attached to an RF electrode carrying member 340. The RF electrode carrying member 340 has four RF electrodes 150A-150D attached thereto, as disclosed in detail hereinabove for the RF electrode carrying member 240 of the Device 200 (it is noted that only two of the RF electrodes 150B and 150C are shown in the cross-sectional view of FIG. 21). The RF electrode carrying member 340 has a sensor passage 345 formed therein for attaching a temperature sensor therein as disclosed in detail hereinabove for the device 200 (the sensor and sensor wire are not shown in FIG. 21 for the sake of clarity of illustration).

RF electrode carrying member 340 has a second passage 343 formed therein for attaching an end of a hollow guiding member 325 included in the RF electrode assembly 330. The guiding member 325 has a first open end 325A attached within the part of the compartment 315 proximal to the RF electrode assembly 330 and a second open end 325B which is attached within the second passage 343 formed in the RF electrode carrying member 340.

The guiding member 325 is shaped as a flexible hollow tube having a lumen open on both sides 325A and 325B. The guiding member 325 passes through an opening 347 formed in the base plate 370 and allows free rotation of the eccentrically attached RF electrode carrying member 340 when the motor 122 is energized.

To operate the device 300, the user opens the openable cover 302B to allow access to the compartment 315. The user inserts the squeezable reservoir 328 filled with the depilatory substance 34 into the compartment 315 and inserts the flexible dispensing hollow tube 328A into the opening at the end 325A of the hollow guiding member 325. The user pushes the dispensing hollow tube 328A into the guiding member 325 until the end of the dispensing hollow tube 328A reaches the open end 325B of the guiding member 325 and is in a suitable position to dispense depilatory substance 34 onto the skin. The user then closes the cover 302B.

When the cover 302B is closed an end 360B of a spring loaded flow regulating lever 360 is pushed against the wall of the guiding member 325 to constrict and close the lumen of the guiding member 325 closing also the part of the dispensing hollow tube 328A which is disposed within the constricted part of the guiding member 325, thus preventing the passage of any of the depilatory substance 34 through the dispensing tube 328A. The spring loaded lever 360 is attached to a spring 364 which is also attached to the wall of the cover 302B. The spring 364 holds the lever 360 in a position of applying pressure on and constricting the lumen of the guiding member 325 until manual pressure is applied to the end 325A of the flow regulating lever 325 by the user resulting in the opening of the constricted lumen of the guiding member 325 and allowing flow of the depilatory substance 34 through the hollow flexible dispensing tube 328A of the squeezable reservoir 328.

When the cover 302B is closed the pressure plate 324 applies pressure on the squeezable reservoir 328 which is squeezed between the fixed printed circuit board 322 and the pressure plate 324 by the forces of the leaf spring 326. Thus, after closing and latching the cover 302B, the depilatory substance 34 in the squeezable reservoir 328 is constantly under pressure but is prevented from flowing through the flexible dispensing hollow tube 328A because of the constricting action of the flow regulating lever 360. When the user presses the end 360A of the flow regulating lever 360 towards the cover 302B, the lever 325 relaxes the constriction of the lumen of the guiding member 325 and the depilatory substance 34 flows from the squeezable reservoir 328 through the flexible dispensing hollow tube 328A and is dispensed onto the surface of the skin adjacent the opening 343 in the RF electrode carrying member 340.

The device 300 also includes a stabilizing member 380 operative as explained hereinabove for the stabilizing member 180 of FIG. 17.

The device 300 also includes the flexible sleeve-like member 205 as disclosed hereinabove and illustrated in FIG. 17. However, to preserve clarity of illustration, the flexible sleeve-like member 205 of the device 300 is not shown in the cross-sectional view of FIG. 21. The end 205B (see FIG. 17) of the flexible sleeve-like member 205 is sealingly attached to the base plate 370 and the end 205A of the flexible sleeve-like member 205 is sealingly attached to the circumferential slot 341 of the RF electrode carrying member 340.

The printed circuit board 322 has an electrical connector 367 which allows electrical connecting of components of the printed circuit board 322 through a mating connector 369 and a cable 390 to any System components disposed outside the device 300. For example, in accordance with one embodiment of the device 300, an external RF energy source (not shown) and an external the DC power source (not shown) may be connected to the device 300 through the cable 390 for providing energy to the RF electrodes 150A-150D and for providing DC current to the motor 122, respectively.

In accordance with another embodiment of the device 300, the RF energy circuitry may be included in the printed circuit board 322, and the cable 390 may supply DC power from an external power supply (not shown) for operating the RF current generating circuitry (not shown in detail) of the printed circuit board 322 and the motor 122.

In accordance with yet another embodiment of the device, all the electronic circuitry for the RF energy source, the motor controlling circuitry, and the circuitry for receiving and processing the signals of the temperature sensor(s) included in the device may be included in the printed circuit board 322. In such a case, an internal power source (not shown in FIG. 21), such as one or more battery, electrochemical cell, rechargeable cell, fuel cell or any other type of electrical energy source known in the art may be internally disposed within the housing 302 of the device 300 (in such a case, the optional connector 367 and the optional cable 390 are redundant and are not included in the device).

When the inventors of the present application first conceived the idea of using the combination of application of heat and depilatory substance for hair removal, they initially tried to apply heat to a depilatory substance treated skin. However, surprisingly, the results of simply applying a non-RF type heating device to skin which has been treated with a depilatory substance were problematic and were qualitatively and quantitatively different than the results obtained later by using an RF energy source for treating the skin.

### Experiment 3

In the first part of this experiment a small area of approximately 16 square centimeters on the outer thigh of the right leg of a male subject was treated with VEET® Sensitive Formula Hair Removal Lotion (commercially available from RECKITT BENCKISER, UK) and then heated for 1 minute to a temperature of approximately 40° C. degrees centigrade for the duration of the treatment by applying a Deep Heat Massager Model DH68-L (commercially available from Kolvin Industries Ltd., Hong Kong, China) to the depilatory gel treated region. The skin temperature was monitored during the treatment by using a PRECISE Model ST652 infra-red thermometer. After the heat treatment, the treated skin area was visually observed to be injured. The treated skin region was undesirably affected by the treatment, and the treated skin region showed bruises which led to the peeling of the skin and bleeding from the bruised treated skin region.

The second part of the experiment was then performed on a similarly sized skin region outer thigh of the left leg of the same male subject using the same depilatory substance (VEET® Sensitive Formula Hair Removal Lotion) but with the heating performed using the same hand held RF device described in EXPERIMENT 1 at a power rate of approximately 6.7 watt for one minute. The skin temperature was monitored during the RF treatment by using the same PRECISE Model ST652 infra-red thermometer as disclosed hereinabove for the first part of the experiment, and was determined to be about 40° C. for the duration of the RF treatment. In contrast to the skin damage observed in the first part of the experiment, the skin region treated with the RF device was not injured and selective hair removal was observed especially near the hair shafts and the hair follicle regions. No bruises or significant skin damage or skin peeling or bleeding were observed in the RF heated depilatory treated skin region on.

Upon further observation (under magnification) of the skin region treated by the combination of depilatory substance application and RF energy application it was unexpectedly and surprisingly observed that the main effects of the depilatory composition were selectively affecting the vicinity of the hair shafts and hair follicles, with almost no visibly discernible effect on other skin. Thus, apparently, applying RF energy to the skin may preferentially (or selectively) heat at least the skin tissues in the vicinity of hair shafts of the skin and the hair shafts to selectively increase the effect of the depilatory substance on the skin tissues and on the hair shafts.

Applying RF energy to the skin may therefore selectively heat skin tissues in the vicinity of the hair shafts of the skin to selectively increase the effect of the depilatory substance on those skin tissues close or adjacent to the hair shafts.

While the reasons for this unexpected and surprising selective effects of RF heating in combination with the depilatory substance, as compared to the application of non-RF caused heating of a depilatory substance treated skin are not presently clearly understood, it may be possible that this effect results from an effect similar (though not necessarily identical) to the selective heat propagation along hair shafts during RF induced heating of hair shafts cut by a hot wire cutting element as described in Published International Patent Application, International Publication Number WO 2007/088541 to Azar et al., incorporated herein by reference in its entirety.

Further experiments were performed to quantitative assess the differences between hair removal achieved by regular shaving of the skin and hair removal achieved by the method of combining applying of a depilatory substance and RF energy application to the skin.

### Experiment 4

The right and left legs of an adult male subject were used for comparing the effects of regular shaving (prior art) to the effects of the depilatory treatment combined with application of RF energy of the present application. Two small skin regions (each having an area of approximately four square centimeters) positioned similarly on the outer thigh of each of the right and left leg of the subject were used as test regions in the experiment.

The first test region on the right leg was treated by applying VEET® Sensitive Formula Hair Removal Lotion (commercially available from RECKITT BENCKISER, UK) to the tested skin region and using the RF device described in detail in EXPERIMENT 1 hereinabove to apply RF energy to the skin for one minute at a power rate of about 6.7 watt, as disclosed in detail in EXPERIMENT 1 hereinabove.

The second test region on the left leg was treated by shaving the test region using a model Gillette Blue II EXCEL shaving razor, commercially available from Gillette a unit of Procter & Gamble, USA. (using a shaving cream commercially available from Gillette, a unit of Procter & Gamble, USA.)

The treated skin regions were then rinsed with tap water to remove the depilatory substance and the shaving gel, dried, and then evaluated with a Visiometer system for measurement of various skin properties and parameters (Skin Visiometer, model SV 600 FireWire, commercially available from CK electronic GmbH, Germany (Courage & Khazaka, Cologne, Germany).

The following skin parameters were evaluated: Surface, Volume, Ser, SEsc and Sew (the definitions of these parameters, as defined by the manufacturer of the Visiometer System, are provided hereinafter. The results of the measured skin parameters for the two different test regions are summarized in TABLE 2.

TABLE 2

| | Surface | Volume | SEr | SEsc | SEw |
|---|---|---|---|---|---|
| RF energy + | 415 | 37 | 6.27 | 0.67 | 125.9 |
| Depilatory | | | | | |
| (Right leg) | | | | | |
| Shaving | 492.1 | 72 | 8.99 | 1.18 | 212.2 |
| (Left Leg) | | | | | |

The following text (placed between quotation marks) was copied from the user manual of the Skin-Visiometer SV 600 FireWire instrument, copied from the user manual supplied with the Visiometer device and labeled (sv600 English FireWire 03/2006 DK), and includes the manufacturers definitions of the terms used in measurements performed:

### “Acronyms and Abbreviations

### Surface (Measurements on the Skin Surface)

Calculates the size of the “wavy” surface in comparison to the stretched (“ironed”) surface (x:1). The smoother the area was before stretching it, the closer the two values are together. The result is displayed in the result window “Surface” in % (e.g. 113=the stretched area is 13% larger than the original surface).

### Volume

Volume calculates the virtual amount of liquid needed in the calculation area to fill the image until the average height of all mountains. The smoother an area before filling up, the less virtual liquid is needed. The result is shown in the result window in the field “Volume” in mm³.

### SELS Parameters

The SELS calculations (Surface Evaluation of Living Skin) consists of following parameters:

### Sesc Scaliness Calculates the Portion of Bright Pixels

Number of pixels where the gray level is higher than the threshold of SEsc.

The smaller SEsc, the less desquamation on the stratum corneum, this corresponds with higher skin moisture.

### SEr Roughness Calculates the Portion of Dark Pixels.

SEr and SEw are depending on the number and width of the wrinkles.

SEr is the roughness parameter and calculates the gray levels above the threshold in comparison to the whole image.

The smaller this value, the less rough the skin.

### SEw Wrinkles Proportional to Number and Width of the Wrinkles

The smaller this value, the less wrinkles measured on the skin.”

It is noted that the results summarized in TABLE 2 indicate that all the determined skin parameters including: Surface, Volume, SEr (representing the skin Roughness), SEsc (representing the skin's scaliness) and SEw (representing a quantitative measure of the skin wrinkles) showed significantly lower values in the test region treated with RF energy application and depilatory treatment, as compared to the respective scores of the razor shaved tested skin region. This indicates that the first test region (RF+Depilatory treatment) exhibited a significantly enhanced smoothness, reduced roughness, reduced wrinkles, reduced desquamation as compared to the razor shaved skin region.

In addition to the comparison of the measured skin parameters of two differently treated skin regions using the VISIOMETER system as described hereinabove, the tested regions were photographed and visually inspected. Reference is now made to FIGS. 21 and 22 are photographs illustrating differences in appearance of human skin after two different skin treatment methods were applied to the skin. FIG. 21 is a microphotograph of part of the first test region (RF energy+Depilatory treatment) and FIG. 22 is a photomicrograph of part of the second test region (razor shaved region).

As may be seen in FIGS. 21-22, the skin appears smoother on the region of the first test region (illustrated in FIG. 21). Additionally the hair stubs seen on FIG. 21 (RF energy+Depilatory treatment) are shorter than those seen in FIG. 22 (Razor Shaved region). As may be seen in FIG. 21, the hair stubs where actually removed to a level below the surface of the skin and are actually disposed within the shaft canal of the hair follicles below the surface of the skin. This is in direct contrast to the hair stubs remaining in the razor shaved test region which are clearly seen in FIG. 22 to protrude as short stubs above the surface of the skin.

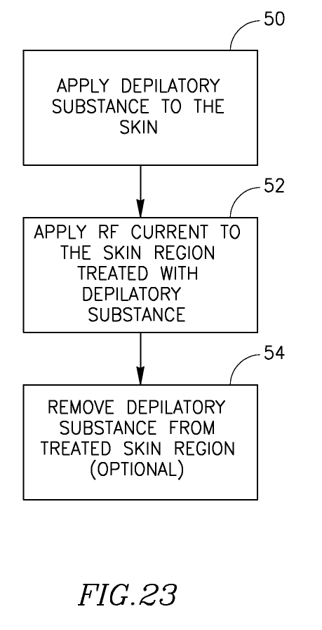

Reference is now made to FIG. 23 which is a schematic flow diagram illustrating the steps of a method of hair removal and/or skin treatment combining the application of a depilatory composition to the skin with the application of RF currents to the treated skin region.

In accordance with an embodiment, the method includes applying a depilatory substance (or a depilatory composition) to the skin (step 50). The application of the depilatory composition or agent or substance may be performed manually as described hereinabove or by using any type of applicator device known in the art, such as but not limited to a, brush, a pad, a spray canister, a squizable container, a pressurized container or any other device known in the art for applying a cream or fluid or gel or solution to the skin.

In accordance with another embodiment of the method, the application of the depilatory substance may also be done automatically by dispensing the depilatory substance from an RF applicator as described in detail hereinabove for the different embodiments of the devices 20 and 40.

In one embodiment, the depilatory cream or composition comprises a thiol component. In another embodiment, the cream or composition comprises a hydroxide component, a thiol component, and a ceteareth component. In another embodiment, the depilatory composition comprises water, a lubricant, a hydroxide component, a thiol component, a ceteareth component, a moisturizer, an aloe component and a fragrance. The lubricant may comprise a mineral oil. The hydroxide component includes at least one of a metal hydroxide, a sodium hydroxide, a calcium hydroxide. The thiol component comprises at least one of a metal thioglycolate, a calcium thioglycolate, a sodium thioglycolate and a potassium thioglycolate. The ceteareth component comprises at least one of a cetearylalcohol and a ceteareth-20. The moisturizer may comprise lanolin. The aloe component may comprise at least one of an aloe juice, an aloe extract and an aloe vera gel.

In another embodiment, the depilatory composition comprises water, a mineral oil, a calcium hydroxide, a ceaterylalcohol, a calcium thioglycolate, a sodium thioglycolate, a ceteareth-20, a lanolin, and an aloe juice and a fragrance. The proportions of the ingredients are similar to that of NAIR®, a hair removal product sold by Carter-Wallace, Inc. In another preferred embodiment, the composition is NAIR®.

In another embodiment, the composition further comprises sodium silicate, a stearyl alcohol, a squalane, a tocopheryl acetate (vitamin E), an avocado oil, a jojoba oil, a bisabol, a sodium lauryl sulfate, a urea, and an iron oxide. The depilatory composition may also include ingredients such as mineral oils and plant extracts.

In one embodiment, the depilatory agent is a compound capable of removing or destroying hair, such as a compound capable of reacting with disulfide bonds of keratin. Examples of such depilatory agents include, but are not limited to, (I) compounds containing one or more thiol groups, such as thiol containing amino acids, and (II) sulfides. Nonlimiting examples of thiol containing compounds include thioglycolic acid, thioethylene glycol, thioglycerol, thioethanol, thioactic acid, thiosalicylic acid and salts thereof (e.g., calcium, sodium, strontium, potassium, ammonium, lithium, magnesium, and other metal salts). Nonlimiting examples of thio-containing amino acids or their derivatives include L-cysteine, D-cysteine, DL-cysteine, N-acetyl-L-cysteine, DL-homocysteine, N-carbamoyl cysteine, glutathion, and cysteamine, and salts and esters thereof (e.g., methyl and ethyl esters). Non-limiting examples of sulfides include calcium sulfide, sodium sulfide, potassium sulfide, lithium sulfide, and strontium sulfide.

RF currents are then applied to the skin region treated with the depilatory substance or depilatory composition (step 52). The application of RF currents to the skin may be done using any of the types of RF current applicators disclosed hereinabove or known in the art. The RF currents are applied to the depilatory substance treated skin for a period of time sufficient for heating the skin or specific parts of the skin in the vicinity of the hair shafts to a temperature which is sufficient for substantially increasing or augmenting or accelerating the chemical depilatory action of the depilatory substance on the hair shafts and/or on the skin tissue adjacent the hair shaft and or the follicular tissues, as compared to the chemical depilatory action of the same depilatory substance in the absence of the RF current application induced temperature increase.

Typically, the time of application of the RF currents to the depilatory substance treated skin is in the order of a few seconds to a few minutes (however, this time may vary as disclosed in more detail hereinafter). Thus, a possible advantage of the methods of the present invention is that the user may save time, since the amount of time required for performing depilation by the methods of the invention may be significantly shortened as compared to using the same depilatory agent without the application of the RF currents to the skin.

However, the time of application of the RF currents to the depilatory substance treated skin may vary considerably depending, inter alia, on the exact chemical composition of the depilatory substance, the concentration and type of the active ingredient(s) in the depilatory composition, the nature of the applied RF current's parameters (including but not limited to the RF current frequency, peak-to peak voltage applied to the RF electrodes, the duty cycle of the RF current delivery and the like), the type of skin being treated (facial skin, limb skin, torso skin and the like) and other factors. Thus, it may be possible to use a treatment time which is substantially higher than a few minutes by using depilatory agents having less active or more gentle depilatory activity which may allow the extension of the RF current treatment of the depilatory agent treated skin beyond the above indicated time values and possibly extending to treatment times of fifteen minutes or longer than fifteen minutes.

The method may also (optionally) include the removing of the remaining depilatory substance (or depilatory composition) from the treated skin region (step 54) after the termination of the application of RF currents to the skin. The (optional) step of removing may be performed by washing or rinsing the treated skin region with water and/or by any other suitable solvent or solvent mixture. For example, it may be possible to remove the remaining depilatory substance by rinsing with water followed by application of a solution containing aloe vera gel or extract for soothing the skin or any other skin soothing agent known in the art. Alternatively, the remaining depilatory agent may be removed from the skin by wiping the treated skin region with a damp cloth or paper towel or by any suitable wiping tissue or piece of absorbent material. Such wiping material may be moistened by water, another solvent or by a solution or cream which is formulated for removing (by absorption) the remaining depilatory substance from the skin or for chemically deactivating the depilatory agents included therein.

For example, as many of the commercially available depilatory formulations are known to contain basic substances such as metal hydroxides and often have very basic pH values (typically in the pH 9-11 range). Therefore, the wiping agent or formulation used for stopping or at least reducing the depilatory chemical action of any amounts of depilatory agents remaining after the wiping or rinsing of the treated skin region may contain one or more cosmetically acceptable buffering or neutralizing agents to readjust the pH of the treated skin region to pH values of normal human skin or even below the normal values (typically, but not obligatorily, pH values in the range of 6.0-7.5 may be acceptable, but other pH values may also be used).

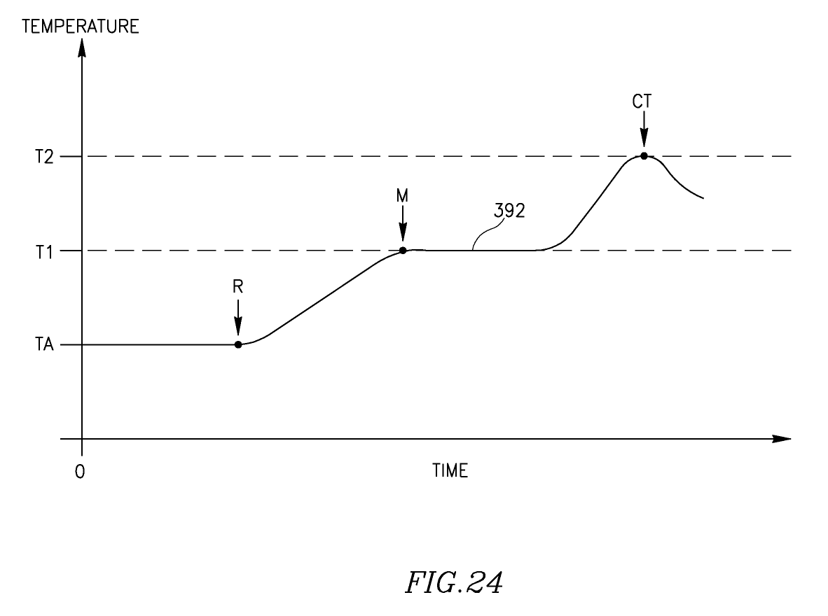

Reference is now made to FIG. 24 which is a schematic graph illustrating a curve representing the skin temperature as a function of time during various stages of operation of skin treating devices and systems of the present application.

The horizontal axis of the graph represents time in arbitrary units and the vertical axis of the graph represents the temperature (in arbitrary units) as measured by a temperature sensor included in any of the skin treating devices and/or systems disclosed herein. The temperature curve 392 schematically represents the skin temperature (such as for example the skin temperature measured by the temperature sensor 250 of FIG. 19), or the temperature of an RF electrode (not shown) as may be measured by a temperature sensor thermally coupled to an RF electrode, in accordance with the different embodiments of the devices used. It is noted that the temperature curve 392 does not represent an actual temperature measured in an experiment but is merely a fictional curve useful as a representation or simulation for demonstrating possible steps (or stages) in the operation of the skin treating devices and systems disclosed herein.

In operation of the device and/or system, (such as but not limited to, any of the devices and/or described herein and including one or more temperature sensors thermally couplable to the skin or thermally coupled to one or more RF electrode(s)), after the application of the depilatory substance to the skin, the RF energy applying device is placed in contact with the depilatory substance treated skin. Before activation of the device, the temperature of the skin TA is the ambient skin temperature. At the time point R the user initiates the RF energy application by the device (such as, for example, by pressing the activating button 204 of the device 200). After the activation, the device starts applying RF energy to the skin (if the device is includes a motorized RF electrode assembly, the device may also initiate the moving and/or rotation of any movable RF electrodes included in the device being used). The temperature increases over time as the application of RF energy to the skin raises the temperature of the skin (as well as the temperature of the RF electrodes). When the temperature reaches a temperature threshold value T1, schematically represented by the point M on the curve 392, the system or the device being used may provide a first signal or indication to the user. The first signal may be an auditory signal such as, for example, a beep or a tone and the like but may also be a visual signal such as, but not limiting to the turning on of a green colored light emitting diode (LED) installed in the device or in the system (LED is not shown). The first signal indicates to the user that the skin (and/or the RF electrodes) have reached the temperature value T1 which is optimal for the skin treating operation of the device. This may indicate to the user that the user may start moving the device along the skin for treating different skin regions.

If, for any reason, the temperature of the skin (and/or of an RF electrode) increases beyond a second threshold value T2 determined as not safe for skin treatment for an extended time period (such as, but not limited to, an instrument malfunction, a stopping of the movement of the electrodes on the skin surface because of a mechanical obstruction or a motor malfunction, or for any other reason) as represented by the point CT on the curve 392, the device may automatically terminate the application of RF energy to the skin through the RF electrodes and may also (optionally) terminate the movement of any movable RF electrodes included in the device (such as, for example by switching of the power supplied to the motor 122, or the like). Additionally and/or alternatively, if the temperature exceeds the value T2, the device and/or system may provide another signal to the user indicating that the use of the device should be terminated (for example, by lifting the device off the skin's surface to reduce or prevent any possible skin damage). The second signal may be auditory and/or visual, such as for example for example, a beep (preferably different then the beep used in the first auditory signal) or a tone (preferably different than the tone used in the first auditory signal) and the like but may also be a visual signal such as, but not limiting to the turning on of a red colored light emitting diode (LED) installed in the device or in the system (LED is not shown). After the automatic termination of the power delivered to the RF electrode, the skin temperature (and/or the RF electrode(s) temperature) starts decreasing as is shown in the later part of the curve 392 after the point CT.

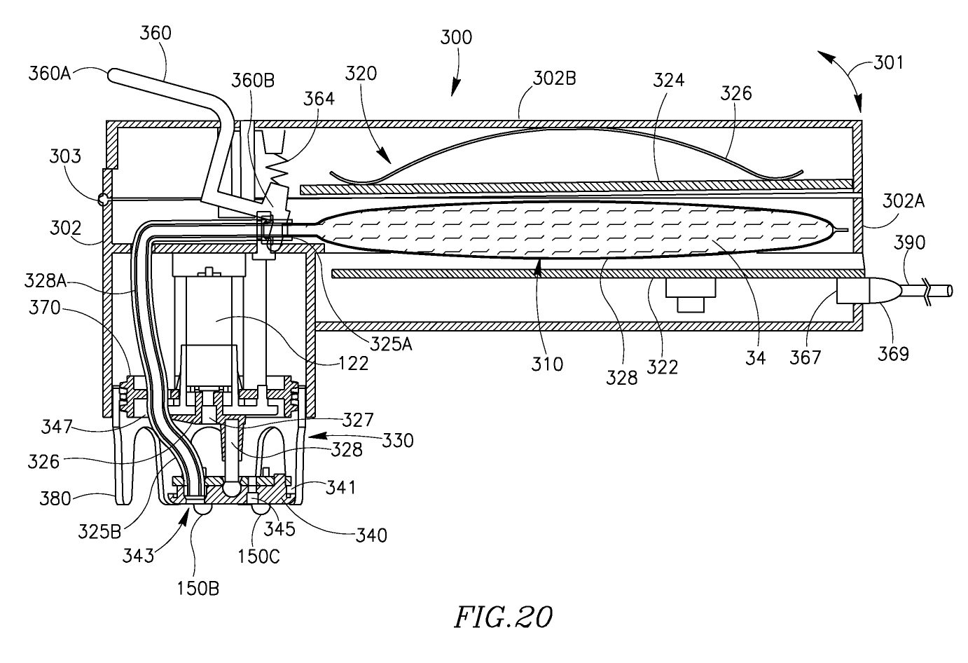

It is noted that the controlling of the activation and termination of RF energy delivery from the RF energy source to the RF electrodes of the Devices and systems may be controlled by a suitable control unit (not shown in detail in the Figures) and/or controller unit that may be included in the device. For example, the controller may be implemented as a microprocessor or microcontroller unit attached to the printed circuit board 322 of the device 300 (of FIG. 20). However, such a controller or processor unit may also be placed in any other desired part of the system (such as, for example, in the RF current generating unit 12 of the system 10 (of FIG. 7).

Additionally, such microcontroller or microprocessor units included in the devices and systems disclosed in the present application may also be used to control the movement of any moving mechanisms used in the devices (such as, but not limited to the motor 122), and (optionally) for controlling the delivering of the first and second signals to the user by using any suitable auditory signal producing device or unit (such as, but not limited to a suitable speaker and/or beeper, and/or any other type or sound generating device known in the art) and/or any suitable visual signal producing device or unit (such as but not limited to LEDs, display units, screens and the like).

It will be appreciated that the methods and devices disclosed herein are not intended to be limited by the particular examples disclosed herein by way of example only. For example, the RF currents may be applied to the skin by using any type of RF current delivery device or system known in the art (including, but not limited to, any such RF current delivery devices designed for skin treatment applications that do not involve hair removal by the use of depilatory compositions as disclosed herein). Furthermore, any RF current delivery device or system having RF electrode shape, number, configurations and implementation (including, but not limited to, stationary RF electrodes, movable RF electrodes, monopolar electrode configurations, bipolar and/or multipolar RF electrode configurations and the like) different than the limited exemplary RF current delivery devices disclosed herein, may also be used in implementing various embodiments of the invention.

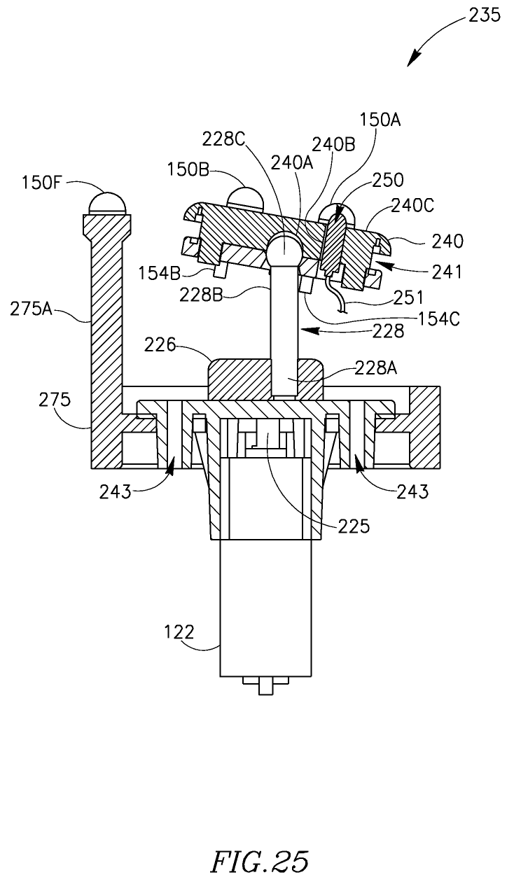

Reference is now made to FIG. 25 which is a schematic part cross-sectional diagram illustrating in detail another motorized movable RF electrode assembly having movable and stationary RF electrodes, suitable for use with the skin treatment device of FIG. 18, in accordance with another embodiment of the skin treatment devices of the present application.

The motorized RF electrode assembly 235 is similar (but not identical) in construction to the motorized RF electrode assembly 230 of FIG. 19, the difference being that the motorized RF electrode assembly 235 includes a base plate 275 different than the base plate 270 of FIG. 19. The base plate 275 has an extending member 275A to which a stationary RF electrode 150F is attached. The extending member 275A has suitable dimensions such that when the motorized RF electrode assembly 235 is attached to the housing 202 and is pushed against the skin, all the RF electrodes including the four movable RF electrodes 150A-150D and the Stationary RF electrode 150F contact the treated skin. It is noted that the insulated electrical wire and the connector electrically coupling the wire to the RF electrode 150F are not shown in FIG. 25 for the sake of clarity of illustration. However, such a wire may pass along and/or may be attached to the extending member 275A or alternatively may pass through a suitable hollow passage (Not shown) passing through the extending member 275A and exiting at the lower side of the base plate 275 (not shown).

In operation, the RF electrodes 150A-150D of the motorized RF Electrode assembly may be rotatably moved along the skin by the RF electrode carrying member 240, as disclosed hereinabove in detail for the motorized RF electrode assembly 230 (of FIG. 19), while the RF electrode 150F does not move and remains stationary with respect to the housing 202. However, it is noted that the RF electrode 150F may also be moved relative to the skin during treatment by simply moving the entire skin treating device (to which the motorized RF electrode assembly 235 is attached) along the surface of the skin.

It will thus be understood by the person skilled in the art that the skin treating devices and systems of the present application may be modified to implement additional embodiments of the devices and systems described herein including any suitable number and configuration of RF electrodes including any suitable or desired combinations of one or more movable RF electrode(s) with One or more Stationary RF electrode(s).

Additionally, it will be appreciated that the specific mechanisms disclosed hereinabove for moving and/or rotating the movable RF electrodes are given by way of example only and any other suitable type of moving and/or rotating mechanism known in the art may be used in the embodiments of the devices and systems disclosed hereinabove.

It is further noted that the dispensing units used in the devices and systems of the present invention for dispensing the depilatory substance onto the skin may include any type of dispensing unit including but not limited to, a hand operated dispensing unit, a pump operated dispensing unit, a valve operated dispensing unit, a pressurized dispensing unit, a propellant operated dispensing unit, a disposable dispensing unit, a replaceable dispensing unit, a refillable dispensing unit, a canister based dispensing unit, a dispensing unit including a fixed reservoir, a dispensing unit including a replaceable reservoir, and a dispensing unit including a disposable reservoir. Additionally, more than one dispensing unit may be included in any of the devices disclosed hereinabove including any combinations of all the different types of dispensing units disclosed hereinabove.

It is finally noted that the springs 154A-154D and/or the flexible sleeve like member 205 disclosed hereinabove may be replaced by any other type of suitable elastic, or spring-like element(s) known in the art, including, but not limited to, a plurality of springs, a plurality of elastic members (such as for example leaf springs or any other type of elastic member known in the art), and a flexible sleeve-like member.

What is claimed is:

1. A device for treatment of skin tissue, the device comprising:
RF electrodes for applying RF energy to the skin; and
a dispensing unit for dispensing a depilatory substance onto the skin.

2. The device according to claim 1 further including an RF energy source for energizing said RF electrodes.

3. The device according to claim 2 wherein said RF energy source is selected from an RF energy source included in said device and an RF energy source disposed outside said device and electrically coupled to said RF electrodes.

4. The device according to claim 1 further including a hand held housing wherein said RF energy source, said RF electrodes and said dispensing unit are attached to said housing.

5. The device according to claim 1 further including a hand held housing said hand held housing is part of a system including an RF current generating unit external to said housing and electrically couplable to said RF electrodes for providing RF energy thereto, and wherein said RF electrodes and said dispensing unit are attached to said hand held housing.

6. The device according to claim 1 wherein said RF electrodes are selected from fixed RF electrodes and movable RF electrodes.

7. The device according to claim 6 wherein said RF electrodes are RF electrodes movable relative to said device and to the surface of the skin for distributing the RF energy to various parts of the skin and for spreading said depilatory substance on the surface of the skin.

8. The device according to claim 7, wherein said RF electrodes are attached to an RF electrode assembly.

9. The device according to claim 8 wherein said RF electrode assembly includes a motor for moving at least one RF electrode of said RF electrodes.

10. The device according to claim 9, wherein said motor is an electrical motor.

11. The device according to claim 9 wherein said RF electrodes are attached to a movable RF electrode carrying member coupled to said motor.

12. The device according to claim 11, also including a stabilizing member attached to said housing for stabilizing the position of said device on the skin while said RF electrode carrying member is moving along the surface of the skin.

13. The device according to claim 12, wherein said stabilizing member has a first portion attached to said housing and a second portion configured for contacting the skin.

14. The device according to claim 13, wherein said second portion of said stabilizing member has a plurality of spaced apart skin contacting portions for contacting the skin.

15. The device according to claim 12, wherein said stabilizing member is detachably attached to said housing.

16. The device according to claim 11, wherein said motor has a rotatable shaft and wherein said RF electrode carrying member is eccentrically attached to said shaft.

17. The device according to claim 11, wherein said RF electrode carrying member is eccentrically attached to a coupling member attached to said rotatable shaft.

18. The device according to claim 11 wherein said motor has a rotatable shaft having a first shaft portion proximal to said motor and a second shaft portion bent at an angle relative to said first shaft portion and wherein said RF electrode carrying member is movably attached to said second shaft portion.

19. The device according to claim 18 wherein said second shaft portion has a spherically shaped member formed at its end distal from said motor, said RF electrode carrying member has a spherically shaped socket formed therein, wherein said spherically shaped member is disposed within said spherically shaped socket to enable said RF electrode carrying member to move at an angle to said second shaft portion.

20. The device according to claim 18 wherein said second shaft portion is movably disposed within a recess formed within said RF electrode carrying member.

21. The device according to claim 11 wherein said RF electrode carrying member is movably attached to said RF electrode assembly or to said housing by one or more spring-like elements.

22. The device according to claim 21 wherein said one or more spring-like members are selected from a plurality of springs, a plurality of elastic members, and a flexible sleeve-like member.

23. The device according to claim 22 wherein said flexible sleeve-like member is a concertina-like member.

24. The device according to claim 22 wherein said one or more spring-like members comprises a flexible sleeve-like member, said flexible sleeve like member has a first end attached to said housing or to said RF electrode assembly and a second end attached to said RF electrode carrying member.

25. The device according to claim 22 wherein said one or more spring-like members are electrically conducting elements electrically connected to said RF electrodes for providing RF energy to said RF electrodes.

26. The device according to claim 1 wherein said dispensing unit is selected from a hand operated dispensing unit, a pump operated dispensing unit, a valve operated dispensing unit, a pressurized dispensing unit, a propellant operated dispensing unit, a disposable dispensing unit, a replaceable dispensing unit, a refillable dispensing unit, a canister based dispensing unit, a dispensing unit including a fixed reservoir, a dispensing unit including a replaceable reservoir, and a dispensing unit including a disposable reservoir.

27. The device according to claim 1 wherein said dispensing unit is fluidically coupled to one or more hollow conduits passing through one or more of said RF electrodes for applying said depilatory substance to the skin through said one or more conduits.

28. The device according to claim 1 wherein said dispensing unit is fluidically coupled to one or more hollow conduits passing through an RF electrode carrying member to which said RF electrodes are attached, for applying said depilatory substance to the skin through said one or more conduits.

29. The device according to claim 1, wherein said dispensing unit comprises a replaceable squeezable reservoir including said depilatory substance and a pressure exerting mechanism configured for applying pressure on said squeezable reservoir.

30. The device according to claim 29, wherein said pressure exerting mechanism comprises a compartment for accommodating said reservoir and a spring loaded pressure plate configured for applying pressure on said squeezable reservoir.

31. The device according to claim 29, wherein said squeezable reservoir comprises a squeezable bag made from a pliable material.

32. The device according to claim 31, wherein said pliable material is a polymer based pliable material.

33. The device according to claim 31, wherein said squeezable reservoir also includes a dispensing tube configured to be sealingly attached to a depilatory substance inlet port included in said dispensing unit.

34. The device according to claim 31, wherein said device includes an RF electrode carrying member to which said RF electrodes are attached, and wherein said squeezable reservoir also includes an elongated flexible hollow dispensing tube configured to be inserted through a hollow guiding member attached to said RF electrode carrying member, said dispensing tube is capable of being inserted into said guiding member and reaching an opening on the surface of said RF electrode carrying member facing said skin, wherein said depilatory substance may be applied to the skin through an opening at the end of said dispensing tube.

35. The device according to claim 34, wherein said dispensing tube further includes a controllably openable valve for controlling the dispensing of said depilatory substance through said dispensing tube.

36. The device according to claim 34, wherein said dispensing tube is an elastic member having a lumen in fluidic communication with said squeezable reservoir and wherein said device also includes a mechanism for constricting or closing the lumen of said dispensing tube by applying pressure on said dispensing tube to regulate or terminate the passage of said depilatory substance through said lumen.

37. The device according to claim 1, also including at least one temperature sensor for determining the temperature of the skin.

38. The device according to claim 37, wherein said at least one temperature sensor is selected from a remote sensing sensor which does not contact the skin, a contact type sensor operable in contact with the skin and any combinations thereof.

39. The device according to claim 37, wherein said temperature sensor is selected from an infra-red detecting temperature sensor, a thermistor based temperature sensor and any combinations thereof.

40. The device according to claim 1, also including at least one temperature sensor for determining the temperature of one or more of said RF electrodes.

41. The device according to claim 40, wherein said at least one temperature sensor is a thermistor.

42. A device for treatment of skin tissue, the device comprising:
means for applying RF energy to the skin; and
means for applying a depilatory substance onto the skin.

43. A device for applying RF energy to skin tissue treated with a depilatory substance, the device comprising:
a housing;
an RF electrode assembly attached to said housing and including RF electrodes for applying RF energy to the skin, at least one of said RF electrodes is movable relative to said housing and to the surface of the skin for distributing the RF energy to various parts of the skin and for spreading said depilatory substance along the surface of the skin; and
an RF energy source for energizing said RF electrodes.

44. The device according to claim 43, wherein said RF energy source is selected from an RF energy source disposed within said housing and electrically connectable to said RF electrodes, and an RF energy source disposed outside of said housing and electrically connectable to said RF electrodes.

45. The device according to claim 43, wherein said RF electrode assembly includes a motor for moving said at least one RF electrode.

46. The device according to claim 45, wherein said motor is an electrical motor.

47. The device according to claim 45, wherein said RF electrodes are attached to a movable RF electrode carrying member coupled to said motor.

48. The device according to claim 47, wherein said motor has a rotatable shaft and wherein said RF electrode carrying member is eccentrically attached to said shaft.

49. The device according to claim 47, wherein said motor has a rotatable shaft having a first shaft portion proximal to said motor and a second shaft portion bent at an angle relative to said first shaft portion and wherein said RF electrode carrying member is attached to said second shaft portion.

50. The device according to claim 49, wherein said second shaft portion has a spherically shaped member formed at its end distal from said motor, said RF electrode carrying member has a spherically shaped socket formed therein, wherein said spherically shaped member is disposed within said spherically shaped socket to enable said RF electrode assembly to move at an angle to said second shaft portion.

51. The device according to claim 47, wherein said electrode carrying member is movably attached to said RF electrode assembly or to said housing by one or more spring-like members.

52. The device according to claim 51, wherein said one or more spring-like members are selected from a plurality of springs, a plurality of elastic members, and a flexible sleeve-like member.

53. The device according to claim 52, wherein said flexible sleeve-like member is a concertina-like member.

54. The device according to claim 52, wherein said one or more spring-like members comprises a flexible sleeve like member, said flexible sleeve like member has a first end sealingly attached to said housing or to said RF electrode assembly and a second end sealingly attached to said RF electrode carrying member.

55. The device according to claim 51, wherein said one or more spring-like members comprises a flexible sleeve-like member, said flexible sleeve like member has a first end attached to said housing or to said RF electrode assembly and a second end attached to said RF electrode carrying member.

56. The device according to claim 51, wherein said one or more spring-like members are electrically conducting elements electrically connected to said RF electrodes for providing RF energy to said RF electrodes.

57. The device according to claim 47, also including a stabilizing member attached to said housing for stabilizing the position of said device on the skin while said RF electrode carrying member is moving along the surface of the skin.

58. The device according to claim 57, wherein said stabilizing member has a first portion attached to said housing and a second portion configured for contacting the skin.

59. The device according to claim 58, wherein said second portion of said stabilizing member has a plurality of spaced apart skin contacting portions for contacting the skin.

60. The device according to claim 57, wherein said stabilizing member is detachably attached to said housing.

61. The device according to claim 43, further including at least one dispensing unit for dispensing a depilatory substance onto the skin.

62. The device according to claim 61 wherein said at least one dispensing unit is selected from a hand operated dispensing unit, a pump operated dispensing unit, a valve operated dispensing unit, a pressurized dispensing unit, a propellant operated dispensing unit, a disposable dispensing unit, a replaceable dispensing unit, a refillable dispensing unit, a canister based dispensing unit, a dispensing unit including a fixed reservoir, a dispensing unit including a replaceable reservoir, and a dispensing unit including a disposable reservoir and any combinations thereof.

63. The device according to claim 62, wherein said at least one dispensing unit comprises a replaceable squeezable reservoir including said depilatory substance and a pressure exerting mechanism configured for applying pressure on said squeezable reservoir.

64. The device according to claim 63, wherein said pressure exerting mechanism comprises a compartment for accommodating said reservoir and a spring loaded pressure plate configured for applying pressure on said squeezable reservoir.

65. The device according to claim 63, wherein said squeezable reservoir comprises a squeezable bag made from a pliable material.

66. The device according to claim 65, wherein said pliable material is a polymer based pliable material.

67. The device according to claim 66, wherein said pliable material is polyethylene.

68. The device according to claim 63, wherein said squeezable reservoir also includes a dispensing tube configured to be attached to a depilatory substance inlet port included in said dispensing unit.

69. The device according to claim 63, wherein said device includes an RF electrode carrying member to which said RF electrodes are attached, and wherein said squeezable reservoir also includes an elongated flexible hollow dispensing tube configured to be inserted through a hollow guiding member attached to said RF electrode carrying member, said dispensing tube is capable of being inserted into said guiding member and reaching an opening on the surface of said RF electrode carrying member facing said skin, wherein said depilatory substance may be applied to the skin through an opening at the end of said dispensing tube.

70. The device according to claim 69, wherein said dispensing tube further includes a controllably openable valve for controlling the dispensing of said depilatory substance through said dispensing tube.

71. The device according to claim 69, wherein said dispensing tube is an elastic member having a lumen in fluidic communication with said squeezable reservoir and wherein said device also includes a mechanism for constricting or closing the lumen of said dispensing tube by applying pressure on said dispensing tube to regulate or terminate the passage of said dispensing member through said lumen.

72. The device according to claim 61 wherein said dispensing unit is fluidically coupled to one or more hollow conduits passing through one or more of said RF electrodes for applying said depilatory substance to the skin through said one or more conduits.

73. The device according to claim 61 wherein said dispensing unit is fluidically coupled to one or more hollow conduits passing through an RF electrode carrying member to which said RF electrodes are attached, for applying said depilatory substance to the skin through said one or more conduits.

74. The device according to claim 43, also including at least one temperature sensor for determining the temperature of the skin.

75. The device according to claim 74, wherein said at least one temperature sensor is selected from a remote sensing type of sensor which does not contact the skin, a contact type sensor operable in contact with the skin and any combinations thereof.

76. The device according to claim 74, wherein said temperature sensor is selected from an infra-red detecting temperature sensor, a thermistor based temperature sensor and any combinations thereof.

77. The device according to claim 43, also including at least one temperature sensor for determining the temperature of one or more of said RF electrodes.

78. The device according to claim 77, wherein said at least one temperature sensor is a thermistor.
